(19) United States
(12) Patent Application Publication
(10) Pub. No.: US 2002/0182677 A1
(43) Pub. Date: Dec. 5, 2002

(54) Pancreatic and ovarian polypeptide, zsig58

(75) Inventors: Paul O. Sheppard, Redmond, WA (US); Yasmin A. Chandrasekher, Mercer Island, WA (US)

Correspondence Address: Jennifer K. Johnson, J.D., ZymoGenetics, Inc., 1201 Eastlake Avenue East, Seattle, WA, 98102, US

(73) Assignee: ZymoGenetics, Inc.

(21) Appl. No.: 10/086,135

(22) Filed: Feb. 26, 2002

Related U.S. Application Data
(62) Division of application No. 09/366,448, filed on Aug. 3, 1999.
(60) Provisional application No. 60/095,199, filed on Aug. 3, 1998.

(51) Int. Cl. C07K 14/705; G06F 19/00; G01N 33/48; C07H 21/04; C12P 21/02; C12N 5/06

(52) U.S. Cl. 435/69.1; 435/320.1; 435/325; 530/350; 536/23.5; 702/19

(57) ABSTRACT

The present invention relates to polynucleotide and polypeptide molecules for zsig58, a novel member of the TTGR family of proteins. The polynucleotides encoding zsig58 may, for example, be used to identify a region of the genome associated with human disease states. The present invention also includes methods for producing the protein, uses therefor and antibodies thereto.

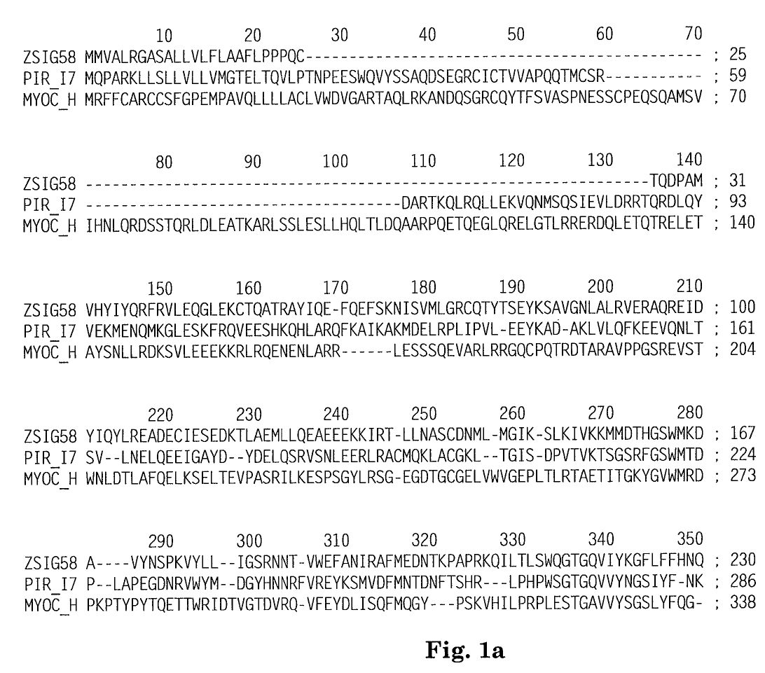

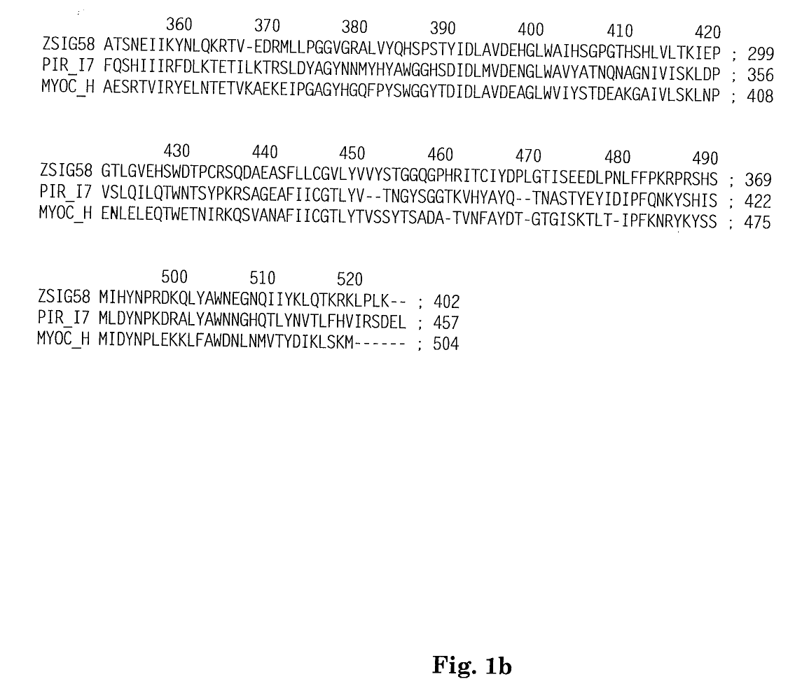

### REFERENCE TO RELATED APPLICATIONS

This application is related to Provisional Application 60/095,199, filed on Aug. 3, 1998. Under 35 U.S.C. §119(e) (1), this application claims benefit of said Provisional Application.

### BACKGROUND OF THE INVENTION

The extracellular matrix, composed of secreted proteins and carbohydrates, has several vital functions. The extracellular matrix functions to bind cells in tissues, to provide a framework for cell movement, and to act as a reservoir for hormones. In addition, secreted proteins that act locally are important in cell interactions. Local interactions of such proteins with a cell result in changes in gene expression leading to a variety of effects including growth and differentiation, cell shape change, and cell movement. For review, see Molecular Cell Biology, Lodish et al., eds., Scientific American Books, New York, 1995, pages 1123-1200.

Trabecular meshwork-induced glucocorticoid response (TIGR) protein is an extracellular protein. Various mutations in TIGR and myocilin genes are associated with primary open angle glaucomas, which are characterized by a common optic neuropathy that can result in total blindness if untreated (Sarfarazi, Human Molecular Genetics, 6:1667-1677, 1997). A cDNA clone was isolated from the ocular ciliary body (Escribano et al., J. Biochem. 118:921-931, 1995). The TIGR gene product is also expressed in trabecular meshwork which includes specialized endothelial cells lining the outflow pathway of the eye. The ocular ciliary body and trabecular meshwork are ocular structures involved in the regulation of intraocular pressure. Major elevations of intraocular pressure are often associated with primary open angle glaucomas. Increased outflow resistance in glaucoma cases were postulated to involve alterations in the type or number of connective tissue components. Studies show that TIGR may be involved in this pathology. TIGR may obstruct aqueous outflow resulting in increased ocular pressure (Stone et al., Science, 275: 668-670, 1997).

Glucocorticoids regulate the induction of TIGR in human trabecular meshwork (H™) cells in culture (Polansky et al., Ophthalmologica, 211:126-139, 1997). The glucocorticoid induction of TIGR was similar to the time course and doses required to cause intraocular pressure in patients treated with corticosteroids, hence providing further support for a role for TIGR in outflow obstruction in glaucoma. Another important observation was the identification of leucine zippers in the TIGR protein structure which explained the occurrence of TIGR-TIGR oligomerization. This finding reinforces the consequences of protein overexpression in disease states. Thus, in addition to genetic alterations, TIGR is regulated by hormonal and environmental stimuli that could result in overexpression of the proteins and resultant disease states.

There is a continuing need to discover new TIGR homologs and the like. The in vivo activity of TIGR illustrate the enormous clinical potential of, and need for, related polypeptides, their agonists, and antagonists. The present invention provides such polypeptides for these and other uses that should be apparent to those skilled in the art from the teachings herein.

### SUMMARY OF THE INVENTION

Within one aspect, the present invention provides an isolated polynucleotide encoding a zsig58 polypeptide comprising a sequence of amino acid residues that is at least 90% identical to an amino acid sequence selected from the group consisting of: (a) the amino acid sequence as shown in SEQ ID NO:2 from amino acid number 141 (Cys) to amino acid number 402 (Lys); (b) the amino acid sequence as shown in SEQ ID NO:2 from amino acid number 26 (Thr) to amino acid number 402 (Lys); and (c) the amino acid sequence as shown in SEQ ID NO:2 from amino acid number 1 (Met) to amino acid number 402 (Lys), wherein the amino acid percent identity is determined using a FASTA program with ktup=1, gap opening penalty=10, gap extension penalty=1, and substitution matrix=BLOSUM62, with other parameters set as default. Within one embodiment, the isolated polynucleotide disclosed above is selected from the group consisting of: (a) a polynucleotide sequence as shown in SEQ ID NO:1 from nucleotide 505 to nucleotide 1290; (b) a polynucleotide sequence as shown in SEQ ID NO:1 from nucleotide 160 to nucleotide 1290; and (c) a polynucleotide sequence as shown in SEQ ID NO:1 from nucleotide 85 to nucleotide 1290. Within another embodiment, the isolated polynucleotide disclosed above comprises nucleotide 1 to nucleotide 1206 of SEQ ID NO:5. Within another embodiment, the isolated polynucleotide disclosed above comprises a sequence of amino acid residues that is identical to an amino acid sequence selected from the group consisting of: (a) the amino acid sequence as shown in SEQ ID NO:2 from amino acid number 141 (Cys) to amino acid number 402 (Lys); (b) the amino acid sequence as shown in SEQ ID NO:2 from amino acid number 26 (Thr) to amino acid number 402 (Lys); and (c) the amino acid sequence as shown in SEQ ID NO:2 from amino acid number 1 (Met) to amino acid number 402 (Lys). Within another embodiment, the isolated polynucleotide disclosed above consists of a sequence of amino acid residues as shown in SEQ ID NO:2 from amino acid number 26 (Thr) to amino acid number 402 (Lys).

Within a second aspect the present invention provides an expression vector comprising the following operably linked elements: a transcription promoter; a DNA segment encoding a zsig58 polypeptide as shown in SEQ ID NO:2 from amino acid number 26 (Thr) to amino acid number 402 (Lys); and a transcription terminator. Within one embodiment, the expression vector disclosed above further comprises a secretory signal sequence operably linked to the DNA segment.

Within a third aspect the present invention provides a cultured cell into which has been introduced an expression vector as disclosed above, wherein the cell expresses a polypeptide encoded by the DNA segment.

Within another aspect the present invention provides a DNA construct encoding a fusion protein, the DNA construct comprising: a first DNA segment encoding a polypeptide selected from the group consisting of: (a) the amino acid sequence of SEQ ID NO: 2 from residue number 1 (Met), to residue 25 (Cys); (b) the amino acid sequence of SEQ ID NO: 2 from residue number 26 (Thr), to residue number 140 (Ser); (c) the amino acid sequence of SEQ ID NO: 2 from residue number 141 (Cys) to amino acid residue 402 (Lys); and (d) the amino acid sequence of SEQ ID NO: 2 from residue number 26 (Thr), to residue number 402 (Lys); and at least one other DNA segment encoding an additional polypeptide, wherein the first and other DNA segments are connected in-frame; and encode the fusion protein.

Within another aspect the present invention provides a fusion protein produced by a method comprising: culturing a host cell into which has been introduced a vector comprising the following operably linked elements: (a) a transcriptional promoter; (b) a DNA construct encoding a fusion protein as disclosed above; and (c) a transcriptional terminator; and recovering the protein encoded by the DNA segment.

Within another aspect the present invention provides an isolated polypeptide comprising a sequence of amino acid residues that is at least 90% identical to an amino acid sequence selected from the group consisting of: (a) the amino acid sequence as shown in SEQ ID NO:2 from amino acid number 141 (Cys) to amino acid number 402 (Lys); (b) the amino acid sequence as shown in SEQ ID NO:2 from amino acid number 26 (Thr) to amino acid number 402 (Lys); and (c) the amino acid sequence as shown in SEQ ID NO:2 from amino acid number 1 (Met) to amino acid number 402 (Lys), wherein the amino acid percent identity is determined using a FASTA program with ktup=1, gap opening penalty=10, gap extension penalty=1, and substitution matrix=BLOSUM62, with other parameters set as default. Within one embodiment the isolate polypeptide disclosed above further contains motifs 1 through 7 spaced apart from N-terminus to C-terminus in a configuration selected from the group consisting of: (a) M1-{46}-M2-{37-41}-M3; and (b) M4-{48-52}-M5-{49}-M1-{1}-M6-{39}-M2-{37-41}-M3-{4}-M7. Within another embodiment the isolate polypeptide disclosed above comprises a sequence of amino acid residues that is identical to an amino acid sequence selected from the group consisting of: (a) the amino acid sequence as shown in SEQ ID NO:2 from amino acid number 141 (Cys) to amino acid number 402 (Lys); (b) the amino acid sequence as shown in SEQ ID NO:2 from amino acid number 26 (Thr) to amino acid number 402 (Lys); and (c) the amino acid sequence as shown in SEQ ID NO:2 from amino acid number 1 (Met) to amino acid number 402 (Lys).

Within another embodiment the isolate polypeptide disclosed above is as shown in SEQ ID NO:2 from amino acid number 26 (Thr) to amino acid number 402 (Lys).

Within another aspect the present invention provides a method of producing a zsig58 polypeptide comprising: culturing a cell as disclosed above; and isolating the zsig58 polypeptide produced by the cell.

Within another aspect the present invention provides a method of detecting, in a test sample, the presence of a modulator of zsig58 protein activity, comprising: transfecting a zsig58-responsive cell, with a reporter gene construct that is responsive to a zsig58-stimulated cellular pathway; and producing a zsig58 polypeptide by the method described above; and adding the zsig58 polypeptide to the cell, in the presence and absence of a test sample; and comparing levels of response to the zsig58 polypeptide, in the presence and absence of the test sample, by a biological or biochemical assay; and determining from the comparison, the presence of the modulator of zsig58 activity in the test sample.

Within another aspect the present invention provides a method of producing an antibody to zsig58 polypeptide comprising the following steps in order: inoculating an animal with a polypeptide selected from the group consisting of: (a) a polypeptide consisting of 9 to 402 amino acids, wherein the polypeptide is identical to a contiguous sequence of amino acids in SEQ ID NO:2 from amino acid number 26 (Thr) to amino acid number 402 (Lys); (b) the amino acid sequence of SEQ ID NO: 2 from residue number 26 (Thr), to residue number 140 (Ser); (c) a polypeptide as disclosed above; and wherein the polypeptide elicits an immune response in the animal; and isolating the antibody from the animal.

Within another aspect, the present invention provides an antibody produced by the method disclosed above, which binds to a zsig58 polypeptide. Within one embodiment, the antibody disclosed above is a monoclonal antibody. Within another aspect, the present invention provides an antibody which specifically binds to a polypeptide as disclosed above.

These and other aspects of the invention will become evident upon reference to the following detailed description of the invention and attached drawings.

### BRIEF DESCRIPTION OF THE DRAWINGS

The FIGURE illustrates an alignment of zsig58 (SEQ ID NO:2), rat neuronal olfactomedin-related ER localized protein (PIR_{—}173639; Danielson et al., J. Neurosci. Res. 38:468-78, 1994) (SEQ ID NO:3), and human TIGR (MYOC_Human; Stone et al., Science 275:668-670, 1997)) (SEQ ID NO:4).

### DETAILED DESCRIPTION OF THE INVENTION

Prior to setting forth the invention in detail, it may be helpful to the understanding thereof to define the following terms:

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “complements of a polynucleotide molecule” denotes a polynucleotide molecule having a complementary base sequence and reverse orientation as compared to a reference sequence. For example, the sequence 5′ ATGCACGGG 3′ is complementary to 5′ CCCGTGCAT 3′.

The term “contig” denotes a polynucleotide that has a contiguous stretch of identical or complementary sequence to another polynucleotide. Contiguous sequences a re said to “overlap” a given stretch of polynucleotide sequence either in their entirety or along a partial stretch of the polynucleotide. For example, representative contigs to the polynucleotide sequence 5′-ATGGAGCTT-3′ are 5′-AGCTTgagt-3′ and 3′-tcgacTACC-5′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

A “DNA construct” is a single or double stranded, linear or circular DNA molecule that comprises segments of DNA combined and juxtaposed in a manner not found in nature. DNA constructs exist as a result of human manipulation, and include clones and other copies of manipulated molecules.

A “DNA segment” is a portion of a larger DNA molecule having specified attributes. For example, a DNA segment encoding a specified polypeptide is a portion of a longer DNA molecule, such as a plasmid or plasmid fragment, that, when read from the 5′ to the 3′ direction, encodes the sequence of amino acids of the specified polypeptide.

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e.greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked,” when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, a-globin, p-globin, and myoglobin are paralogs of each other.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 51 non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel DNA sequence that encodes a polypeptide having homology to human TIGR and its homologs neuronal olfactomedin-related (Genbank Accession No. Q99784), myocilin precursor (Adam et al., Hum. Mol. Genet. 6:2091-7, 1997) and olfactomedin precursor (Bal et al., Biochemistry 32:1047-53, 1993). Analysis of the tissue distribution of the mRNA corresponding to this novel DNA showed that expression was highest in ovary and pancreas. The polypeptide has been designated zsig58.

The novel zsig58 polypeptides of the present invention were initially identified by querying an EST database for proteins homologous to proteins having a secretory signal sequence. These proteins are characterized by an upstream methionine start site and a hydrophobic region of approximately 13 amino acids, followed by a peptide signal peptidase cleavage site. An EST database was queried for novel DNA sequences whose translations would meet these search criteria. An EST was found and its corresponding cDNA was sequenced. The novel polypeptide encoded by the cDNA showed homology with human TIGR (Nguyen, J. Biol. Chem. 273:6341-6350, 1998). Based on TIGR homology, the zsig58 nucleotide sequence is believed to encode the entire coding sequence of the predicted protein. Zsig58 may be a novel cell-cell signaling molecule, growth factor, or secreted extracellular matrix associated protein with growth factor hormone activity, or the like, and is a novel member of the TIGR family of proteins.

The sequence of the zsig58 polypeptide was obtained from a single clone believed to contain its corresponding polynucleotide sequence. The clone was obtained from a pituitary library. Other libraries that might also be searched for such sequences include ovary, placenta, and the like.

The nucleotide sequence of a representative zsig58-encoding DNA is described in SEQ ID NO:l, and its deduced 403 amino acid sequence is described in SEQ ID NO:2. In its entirety, the zsig58 polypeptide (SEQ ID NO:2) represents a full-length polypeptide segment (residue 1 (Met) to residue 402 (Lys) of SEQ ID NO:2) Zsig58 is similar in length, and contains a signal sequence, and a region analogous to TIGR in the carboxy two thirds of the molecule. The domains and structural features of zsig58 are further described below.

Analysis of the zsig58 polypeptide encoded by the DNA sequence of SEQ ID NO:1 revealed an open reading frame encoding 403 amino acids (SEQ ID NO:2) comprising a predicted signal peptide of 25 amino acid residues (residue 1 (Met) to residue 25 (Cys) of SEQ ID NO:2), and a mature polypeptide of 377 amino acids (residue 26 (Thr) to residue 402 (Lys) of SEQ ID NO:2). The zsig58 polypeptide contains a region, referred to hereinafter as the “Carboxy domain,” corresponding to amino acid residue 141 (Cys) to amino acid residue 402 (Lys) of SEQ ID NO:2.

Within the carboxy domain are the several motifs of conserved amino acids based comparison amongst family members (see FIGURE). Moreover, several regions of low variance are also present within the carboxy domain (see, Sheppard, P. et al., Gene 150:163-167, 1994). Examining the multiple alignment (see, FIGURE) revealed the following motifs that are both highly conserved and exhibit low degeneracy:

1) “motif 1” (corresponding to amino acids 271 to 276 of SEQ ID NO:2);

2) “motif 2” (corresponding to amino acids 321 to 328 of SEQ ID NO:2);

3) “motif 3” (corresponding to amino acids 370 to 375 of SEQ ID NO:2); and

Motifs 1 through 3 are spaced apart from N-terminus to C-terminus in a configuration represented by the following:

M1-{46}-M2-{37-41}-M3,

where M# denotes the specific motif disclosed above (e.g., M1 is motif 1, etc.) and

{#} denotes the number of amino acids between the motifs.

In addition, several other conserved or low degeneracy motifs in the zsig44 carboxy domain are evident:

4) “motif 4” (corresponding to amino acids 162 to 167 of SEQ ID NO:2);

5) “motif 5” (corresponding to amino acids 216 to 221 of SEQ ID NO:2);

6) “motif 6” (corresponding to amino acids 278 to 283 of SEQ ID NO:2); and

7) “motif 7” (corresponding to amino acids 380 to 385 of SEQ ID NO:2); and

Motifs 4 through 7 are spaced apart from N-terminus to C-terminus in a configuration represented by the following:

M4-{48-52}-M5-{56}-M6-{92-96}-M7,

where M# denotes the specific motif disclosed above (e.g., M4 is motif 4, etc.) and

{#} denotes the number of amino acids between the motifs.

Motifs 1 through 7 are spaced apart from N-terminus to C-terminus in a configuration represented by the following:

M4-{48-52}-MS-{49}-Ml-{1}-M6-{39}-M2-{37-41}-M3-{4}-M7.

where M# denotes the specific motif disclosed above (e.g., M4 is motif 4, etc.) and

{#} denotes the number of amino acids between the motifs.

The presence of conserved and low variance motifs generally correlates with or defines important structural regions in proteins. Regions of low variance (e.g., hydrophobic clusters) are generally present in regions of structural importance (Sheppard, P. et al., Gene 150:163-167, 1994). Such regions of low variance often contain rare or infrequent amino acids, such as Tryptophan. The regions flanking and between such conserved and low variance motifs may be more variable, but are often functionally significant because they may relate to or define important structures and activities such as binding domains, biological and enzymatic activity, signal transduction, tissue localization domains and the like. For example, the flanking region N-terminal to the carboxy domain, comprising amino acid sequence of SEQ ID NO: 2 from residue number 26 (Thr), to residue number 140 (Ser) may be functionally significant.

In addition, there are several individual conserved amino acids throughout the carboxy domain located in SEQ ID NO:2 at the following amino acid numbers: 141 (Cys), 147 (Gly), 180 (Gly), 186 (Val), 188 (Glu), 195 (Phe), 196 (Met), 209 (Leu), 216 (Thr), 219 (Val), 223 (Gly), 227 (Phe), 233 (Ser), 235 (Ile), 241 (Leu), 245 (Thr), 255 (Gly), 293 (Val), 296 (Lys), 308 (Trp), 310 (Thr), and 391 (Tyr).

There are 3 consensus phosphorylation sites in zsig58 polypeptide: SLK, amino acid 150 (Ser) to amino acid 152 (Lys) of SEQ ID NO:2; SPK, amino acid 172 (Ser) to amino acid 174 (Lys) of SEQ ID NO:2; and, TKR, amino acid 395 (Thr) to amino acid 397 (Arg) of SEQ ID NO:2. These sites are not present in PIR_{—}17 or TIGR, see FIGURE. Moreover, zsig58 polypeptide has predicted glycosylation site located at amino acid numbers 66 (Asn), 137 (Asn), 183 (Asn) in SEQ ID NO:2. Corresponding glycosylation sites at these amino acid positions are not found in TIGR or PIR-173639, see FIGURE. In addition, an endoplasmic reticulum retention signal is not present in zsig58, suggesting that the molecule, unlike TIGR, is secreted from the cell.

The regions of conserved amino acid residues in the carboxy domain, transmembrane domain, or the conserved motifs of zsig58 can be used as tools to identify new family members. For instance, reverse transcription-polymerase chain reaction (RT-PCR) can be used to amplify sequences encoding the conserved regions from RNA obtained from a variety of tissue sources or cell lines. In particular, highly degenerate primers designed from the zsig58 sequences are useful for this purpose. Designing and using such degenerate primers may be readily performed by one of skill in the art.

The present invention also provides polynucleotide molecules, including DNA and RNA molecules, that encode the zsig58 polypeptides disclosed herein. Those skilled in the art will readily recognize that, in view of the degeneracy of the genetic code, considerable sequence variation is possible among these polynucleotide molecules. SEQ ID NO:5 is a degenerate DNA sequence that encompasses all DNAs that encode the zsig58 polypeptide of SEQ ID NO:2. Those skilled in the art will recognize that the degenerate sequence of SEQ ID NO:5 also provides all RIA sequences encoding SEQ ID NO:2 by substituting U for T. Thus, zsig58 polypeptide-encoding polynucleotides comprising nucleotide 1 to nucleotide 1206 of SEQ ID NO:5 and their RNA equivalents are contemplated by the present invention. Table 1 sets forth the one-letter codes used within SEQ ID NO:5 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:5, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” In general, see, Grantham, et al, Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nucl. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid Threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:5 serves as a template for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention the isolated polynucleotides will hybridize to similar sized regions of SEQ ID NO:1, or a sequence complementary thereto, under stringent conditions. In general, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typical stringent conditions are those in which the NaCl concentration is up to about 0.03 M at pH 7 and the temperature is at least about 60° C.

As previously noted, the isolated polynucleotides of the present invention include DNA and RNA. Methods for preparing DNA and RNA are well known in the art. In general, RNA is isolated from a tissue or cell that produces large amounts of zsig58 RNA. Such tissues and cells are identified by Northern blotting (Thomas, Proc. Natl. Acad. Sci. USA 77:5201, 1980), and include pancreas, ovary, and cell lines derived therefrom. Total RNA can be prepared using guanidinium isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. Alternatively, genomic DNA can be isolated. Polynucleotides encoding zsig58 polypeptides are then identified and isolated by, for example, hybridization or PCR.

A full-length clone encoding zsig58 can be obtained by conventional cloning procedures. Complementary DNA (CDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to zsig58, receptor fragments, or other specific binding partners.

The polynucleotides of the present invention can also be synthesized using DNA synthesis machines. Currently the method of choice is the phosphoramidite method. If chemically synthesized double stranded DNA is required for an application such as the synthesis of a gene or a gene fragment, then each complementary strand is made separately. The production of short polynucleotides (60 to 80 bp) is technically straightforward and can be accomplished by synthesizing the complementary strands and then annealing them. However, for producing longer polynucleotides (>300 bp), special strategies are usually employed, because the coupling efficiency of each cycle during chemical DNA synthesis is seldom 100%. To overcome this problem, synthetic genes (double-stranded) are assembled in modular form from single-stranded fragments that are from 20 to 100 nucleotides in length.

One method for building a synthetic gene requires the initial production of a set of overlapping, complementary oligonucleotides, each of which is between 20 to 60 nucleotides long. Each internal section of the gene has complementary 3′ and 5′ terminal extensions designed to base pair precisely with an adjacent section. Thus, after the gene is assembled, process is completed by sealing the nicks along the backbones of the two strands with T4 DNA ligase. In addition to the protein coding sequence, synthetic genes can be designed with terminal sequences that facilitate insertion into a restriction endonuclease site of a cloning vector. Moreover, other sequences should can be added that contain signals for proper initiation and termination of transcription and translation.

An alternative way to prepare a full-length gene is to synthesize a specified set of overlapping oligonucleotides (40 to 100 nucleotides). After the 3′ and 5′ short overlapping complementary regions (6 to 10 nucleotides) are annealed, large gaps still remain, but the short base-paired regions are both long enough and stable enough to hold the structure together. The are gaps filled and the DNA duplex is completed via enzymatic DNA synthesis by E. coli DNA polymerase I. After the enzymatic synthesis is completed, the nicks are sealed with T4 DNA ligase. Double-stranded constructs are sequentially linked to one another to form the entire gene sequence which is verified by DNA sequence analysis. See Glick and Pasternak, Molecular Biotechnology, Principles & Applications of Recombinant DNA, (ASM Press, Washington, D.C. 1994); Itakura et al., Annu. Rev. Biochem. 53: 323-56, 1984 and Climie et al., Proc. Natl. Acad. Sci. USA 87:633-7, 1990.

Zsig58 polynucleotide sequences disclosed herein can also be used as probes or primers to clone 5′ non-coding regions of a zsig58 gene. In view of the tissue-specific expression observed for zsig58 by Northern blotting, this gene region is expected to provide for ovarian or pancreatic-specific expression. Promoter elements from a zsig58 gene could thus be used to direct the tissue-specific expression of heterologous genes in, for example, transgenic animals or patients treated with gene therapy. Cloning of 5′ flanking sequences also facilitates production of zsig58 proteins by “gene activation” as disclosed in U.S. Pat. No. 5,641,670. Briefly, expression of an endogenous zsig58 gene in a cell is altered by introducing into the zsig58 locus a DNA construct comprising at least a targeting sequence, a regulatory sequence, an exon, and an unpaired splice donor site. The targeting sequence is a zsig58 5′ non-coding sequence that permits homologous recombination of the construct with the endogenous zsig58 locus, whereby the sequences within the construct become operably linked with the endogenous zsig58 coding sequence. In this way, an endogenous zsig58 promoter can be replaced or supplemented with other regulatory sequences to provide enhanced, tissue-specific, or otherwise regulated expression.

The present invention further provides counterpart polypeptides and polynucleotides from other species (orthologs). These species include, but are not limited to mammalian, avian, amphibian, reptile, fish, insect and other vertebrate and invertebrate species. Of particular interest are zsig58 polypeptides from other mammalian species, including murine, porcine, ovine, bovine, canine, feline, equine, and other primate polypeptides. Orthologs of human zsig58 can be cloned using information and compositions provided by the present invention in combination with conventional cloning techniques. For example, a cDNA can be cloned using mRNA obtained from a tissue or cell type that expresses zsig58 as disclosed herein. Suitable sources of mRNA can be identified by probing Northern blots with probes designed from the sequences disclosed herein. A library is then prepared from MRNA of a positive tissue or cell line. A zsig58-encoding cDNA can then be isolated by a variety of methods, such as by probing with a complete or partial human CDNA or with one or more sets of degenerate probes based on the disclosed sequences. A cDNA can also be cloned using the polymerase chain reaction (PCR) (Mullis, U.S. Pat. No. 4,683,202), using primers designed from the representative human zsig58 polynucleotidesequence disclosed herein. Within an additional method, the cDNA library can be used to transform or transfect host cells, arid expression of the cDNA of interest can be detected with an antibody to zsig58 polypeptide. Similar techniques can also be applied to the isolation of genomic clones.

Those skilled in the art will recognize that the sequence disclosed in SEQ ID NO:1 represents a single allele of human zsig58 and that allelic variation and alternative splicing are expected to occur. Allelic variants of this sequence can be cloned by probing cDNA or genomic libraries from different individuals according to standard procedures. Allelic variants of the DNA sequence shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the zsig58 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The present invention also provides isolated zsig58 polypeptides that are substantially homologous to the polypeptides of SEQ ID NO:2 and their orthologs. The term “substantially homologous” is used herein to denote polypeptides having 50%, preferably 60%, more preferably at: least 80%, sequence identity to the sequences shown in SEQ ID NO:2 or their orthologs. Such polypeptides will more preferably be at least 90% identical, and most preferably 95%. or more identical to SEQ ID NO:2 or its orthologs). Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{\text{Total number of identical matches}}{\begin{matrix}\text{[length of the longer sequence plus the} \\ \text{number of gaps introduced into the longer} \\ \text{sequence in order to align the two sequences]}\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zsig58. The FASTA algorithm is described by Pearson and Lipman, Proc. Nat'l Acad. Sci. USA 85:2444, 1988; and by Pearson, Meth. Enzymol. 183:63, 1990.

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Preferred parameters for FASTA analysis are: ktup=1, gap opening penalty=10, gap extension penalty=1, and substitution matrix=BLOSUM62, with other parameters set as default. These parameters can be introduced into a FASTA program by modifying the scoring matrix file (“SMATRIX”), as explained in Appendix 2 of Pearson, Meth. Enzymol. 183:63, 1990.

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

The BLOSUM62 table (Table 3) is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed below), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Variant zsig58 polypeptides or substantially homologous zsig58 polypeptides are characterized as having one or more amino acid substitutions, deletions or additions. These changes are preferably of a minor nature, that is conservative amino acid substitutions (see Table 4) and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides of from about 345 to about 435 amino acid residues that comprise a sequence that is at least 80%, preferably at least 90%, and more preferably 95% or more identical to the corresponding region of SEQ ID NO:2. Polypeptides comprising affinity tags can further comprise a proteolytic cleavage site between the zsig58 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

The present invention further provides a variety of other polypeptide fusions and related multimeric proteins comprising one or more polypeptide fusions. For example, a zsig58 polypeptide can be prepared as a fusion to a dimerizing protein as disclosed in U.S. Pat. Nos. 5,155,027 and 5,567,584. Preferred dimerizing proteins in this regard include immunoglobulin constant region domains. Immunoglobulin-zsig58 polypeptide fusions can be expressed in genetically engineered cells to produce a variety of multimeric zsig58 analogs. Auxiliary domains can be fused to zsig58 polypeptides to target them to specific cells, tissues, or macromolecules (e.g., collagen). For example, a zsig58 polypeptide or protein could be targeted to a predetermined cell type by fusing a zsig58 polypeptide to a ligand that specifically binds to a receptor on the surface of the target cell. In this way, polypeptides and proteins can be targeted for therapeutic or diagnostic purposes. A zsig58 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et. al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection. of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for zsig58 amino acid residues.

Essential amino acids in the polypeptides of the present invention can be identified according to procedures known in the art, such as site-directed mutagenesis or alanine-scanning mutagenesis (Cunningham arid Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699-708, 1996. Sites of ligand-receptor or other biological interaction can also be determined by physical analysis of structure, as determined by such techniques as nuclear magnetic resonance, crystallography, eLectron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related proteins, such as the human TIGR.Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed zsig58 DNA and polypeptide sequences can be generated through DNA shuffling as disclosed by Stemmer, Nature 370:389-91, 1994; Stemmer, Proc. Natl. Acad. Sci. USA 91:10747-51, 1994; and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode active polypeptides (e.g., signal transduction, or binding activities) can be recovered from the host cells and rapidly sequenced using modern equipment. These methods allow the rapid determination of the importance of individual amino acid residues in a polypeptide of interest, and can be applied to polypeptides of unknown structure.

Using the methods discussed herein, one of ordinary skill in the art can identify and/or prepare a variety of polypeptide fragments or variants of SEQ ID NO:2 that retain, for example, TIGR-like properties, binding, cell-cell communication, or signal transduction activity of the wild-type zsig58 protein. For example, using the methods described above, one could identify a receptor binding domain on zsig58; heterodimeric and homodimeric binding domains; other functional or structural domains; or other domains important for protein-protein interactions or signal transduction. Such polypeptides may also include additional polypeptide segments, such as affinity tags, as generally disclosed herein.

For any zsig58 polypeptide, including variants and fusion proteins, one of ordinary skill in the art can readily generate a fully degenerate polynucleotide sequence encoding that variant using the information set forth in Tables 1 and 2 above.

The zsig58 polypeptides of the present invention, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a zsigS8 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zsig58 polypeptide into the secretory pathway of a host cell, a secretory signal sequence (also known as a leader sequence, prepro sequence or pre sequence) is provided in the expression vector. The secretory signal sequence may be that of zsig58, or may be derived from another secreted protein (e.g., t-PA) or synthesized de novo. The secretory signal sequence is operably linked to the zsig58 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

Alternatively, the secretory signal sequence contained in the polypeptides of the present invention is used to direct other polypeptides into the secretory pathway. The present invention provides for such fusion polypeptides. A signal fusion polypeptide can be made wherein a secretory signal sequence that encodes a signal peptide from amino acid 1 (Met) to amino acid 25 (Cys) of SEQ ID NO:2 is operably linked to another DNA segment encoding a polypeptide, using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein. Such fusions may be used in vivo or in vitro to direct peptides through the secretory pathway.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Manassas, Va. Other suitable cell lines include but are not limited to ovarian and pancreatic cell lines, osteoblast, osteoclast, hematopoietic cell lines, and lymphoid cell lines. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. The second method of making recombinant zsig58 baculovirus utilizes a transposon-based system described by Luckow (Luckow, V. A, et al., J Virol 67:4566-79, 1993). This system is sold in the Bac-to-Bac™ kit (Life Technologies, Rockville, Md.). This system utilizes a transfer vector, pFastBac1™ (Life Technologies) containing a Tn7 transposon to move the DNA encoding the zsig58 polypeptide into a baculovirus genome maintained in E. coli as a large plasmid called a “bacmid.” The pFastBac1™ transfer vector utilizes the AcNPV polyhedrin promoter to drive the expression of the gene of interest, in this case zsig58. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol. Chem. 270:1543-9, 1995. In such transfer vector constructs, a short or long version of the basic protein promoter can be used. Moreover, transfer vectors can be constructed which replace the native zsig58 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native zsig58 secretory signal sequence. In addition, transfer vectors can include an in-frame fusion with DNA encoding an epitope tag at the C- or N-terminus of the expressed zsig58 polypeptide, for example, a Glu-Glu epitope tag ((Grussenmeyer, T. et al., Proc. Natl. Acad. Sci. 82:7952-4, 1985). Using a technique known in the art, a transfer vector containing zsig58 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zsig58 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the zsig58 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (T) of from 1 to 40 milliseconds, most preferably about 20 milliseconds. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a zsig58 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need f or denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

It is preferred to purify the polypeptides of the present invention to ≧80% purity, more preferably to 90% purity, even more preferably ≧95% purity, and articularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant zsig58 polypeptides (or chimeric zsig58 polypeptides) can be purified using fractionation and/or conventional purification methods and media. For example, the particular purification methods for TIGR, described in Nguyen, supra., are exemplary, and can be adapted to zsig58 polypeptide by one of ordinary skill in the art using methods described below. An exemplary purification method for protein constructs having an affinity tag such as an N-terminal or C-terminal FIAG tag produced from mammalian cells, such as BHK cells, involves using an antibody to the FLAG tag epitope to purify the zsig58 protein. Conditioned media from BHK cells is sequentially sterile filtered through a 4 inch, 0.2 mM Millipore (Bedford, Mass.) OptiCap capsule filter and a 0.2 mM Gelman (Ann Arbor, Mich.) Supercap 50. The material is then concentrated using an Amicon (Beverly, Mass.) DC 10L concentrator fitted with an A/G Tech (Needham, Mass.) hollow fiber cartridge with a 15 sq. ft. 3000 kDa cutoff membrane. The concentrated material is again sterile-filtered with the Gelman filter as described above. A aliquot of anti-Flag Sepharose (Eastman Kodak, Rochester, N.Y.) is added to the sample for batch adsorption and the mixture is gently agitated on a Wheaton (Millville, N.J.) roller culture apparatus for 18.0 h at 4° C.

The mixture is then poured into a 5.0×20.0 cm Econo-Column (Bio-Rad, Laboratories, Hercules, Calif.) and the gel is washed with 30 column volumes of phosphate buffered saline (PBS). The unretained flow-through fraction is discarded. Once the absorbance of the effluent at 280 nM is less than 0.05, flow through the column is reduced to zero and the anti-Flag Sepharose gel is washed with 2.0 column volumes of PBS containing 0.2 mg/ml of Flag peptide, N-Asp-Tyr-Lys-Asp-Asp-Asp-Asp-Lys-C (SEQ ID NO:6) (Eastman Kodak). After 1.0 hour at 4° C., flow is resumed and the eluted protein is collected. This fraction is referred to as the peptide elution. The anti-Flag Sepharose gel is washed with 2.0 column volumes of 0.1 M glycine, pH 2.5, and the glycine wash is collected separately. The pH of the glycine-eluted fraction is adjusted to 7.0 by the addition of a small volume of 10× PBS and stored at 4° C.

The peptide elution is concentrated to 5.0 ml using a 5,000 molecular weight cutoff membrane concentrator (Millipore, Bedford, Mass.) according to the manufacturer's instructions. The concentrated peptide elution is then separated from free peptide by chromatography on a 1.5×50 cm Sephadex G-50 (Pharmacia, Piscataway, N.J.) column equilibrated in PBS at a flow rate of 1.0 ml/min using a BioCad Sprint HPLC system (PerSeptive BioSystems, Framingham, Mass.). Two-ml fractions are collected and the absorbance at 280 nM is monitored. The first peak of material absorbing at 280 nM and eluting near the void volume of the column is collected.

SDS-PAGE, Western analysis, amino acid analysis and N-terminal sequencing can be done to the purified protein. Protein concentration can be determined by BCA analysis (Pierce, Rockford, Ill.).

Ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their biochemical, structural, and biological properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Within additional embodiments of the invention, a fusion of the polypeptide of interest and an affinity tag (e.g., maltose-binding protein, his-tag, or immunoglobulin domain) may be constructed to facilitate purification.

Moreover, using methods described in the art, polypeptide fusions, or hybrid zsig58 proteins, are constructed using regions or domains of zsig58 polypeptide in combination with those of other related proteins (e.g. human TIGR), or heterologous proteins (Sambrook et al., ibid., Altschul et al., ibid., Picard, Cur. Olin. Biology, 5:511-5, 1994, and references therein). These methods allow the determination of the biological importance of larger domains or regions in a polypeptide of interest. Such hybrids may alter reaction kinetics, binding, constrict or expand the substrate specificity, or alter tissue and cellular localization of a polypeptide, and can be applied to polypeptides of unknown structure.

Fusion proteins can be prepared by methods known to those skilled in the art by preparing each component of the fusion protein and chemically conjugating them. Alternatively, a polynucleotide encoding different components of the fusion protein in the proper reading frame can be generated using known techniques and expressed by the methods described herein. For example, part or all of one or more domains conferring a biological function may be swapped between zsig58 of the present invention with the corresponding domain(s) from another protein family member, such as TIGR. Such domains include, but are not limited to, the secretory signal sequence, conserved motifs (e.g., motifs 1 through 7), variable regions between conserved motifs, the flanking region N-terminal to the carboxy domain, and the carboxy domain. Such fusion proteins would be expected to have a biological functional profile that is the same or similar to polypeptides of the present invention or other TIGR family proteins, depending on the fusion constructed. Moreover, such fusion proteins may exhibit other properties as disclosed herein.

Standard molecular biological and cloning techniques can be used to swap the equivalent domains between the zsig58 polypeptide and those polypeptides to which they are fused. Generally, a DNA segment that encodes a domain of interest, e.g., a zsig58 domain described herein, is operably linked in frame to at least one other DNA segment encoding an additional polypeptide (for instance a domain or region from TIGR or similar polypeptide), and inserted into an appropriate expression vector, as described herein. Generally DNA constructs are made such that the several DNA segments that encode the corresponding regions of a polypeptide are operably linked in frame to make a single construct that encodes the entire fusion protein, or a functional portion thereof. For example, a DNA construct would encode from N-terminus to C-terminus a fusion protein comprising a secretory signal sequence, followed by conserved motifs (e.g., motifs 1 through 7 with variable regions between conserved motifs), followed by a flanking region N-terminal to the carboxy domain, and a carboxy domain. Such fusion proteins can be expressed, isolated, and assayed for activity as described herein.

zsig58 polypeptides or fragments thereof may also be prepared through chemical synthesis. zsig58 polypeptides may be monomers or multimers; glycosylated or non-glycosylated; pegylated or non-pegylated; and may or may not include an initial methionine amino acid residue.

Polypeptides of the present invention can also be synthesized by exclusive solid phase synthesis, partial solid phase methods, fragment condensation or classical solution synthesis. Methods for synthesizing polypeptides are well known in the art. See, for example, Merrifield, J. Am. Chem. Soc. 85:2149, 1963; Kaiser et al., Anal. Biochem. 34:595, 1970. After the entire synthesis of the desired peptide on a solid support, the peptide-resin is with a reagent which cleaves the polypeptide from the resin and removes most of the side-chain protecting groups. Such methods are well established in the art.The activity of molecules of the present invention can be measured using a variety of assays that measure for example, signal transduction, cell motility, steroidogenesis, mitogenesis or binding. Such assays are well known in the art.

The zsig58 polypeptides of the present invention can be used to study pancreatic cell proliferation or differentiation. Such methods of the present invention generally comprise incubating α cells, β cells, δ cells, F cells and acinar cells in the presence and absence of zsig58 polypeptide, monoclonal antibody, agonist or antagonist thereof and observing changes in islet cell proliferation or differentiation.

A further aspect of the invention provides a method for studying insulin. Such methods of the present invention comprise incubating adipocytes in a culture medium comprising zsig58 polypeptide, monoclonal antibody, agonist or antagonist thereof±insulin and observing changes in adipocyte protein secretion or differentiation.

The present invention also provides methods of studying mammalian cellular metabolism. Such methods of the present invention comprise incubating cells to be studied, for example, an appropriate human cell line, ±zsig58 polypeptide, monoclonal antibody, agonist or antagonist thereof, and observing changes in adipogenesis, gluconeogenesis, glycogenolysis, lipogenesis, glucose uptake, or the like.

Also, zsig58 polypeptides, agonists or antagonists thereof may be therapeutically useful for promoting wound healing, for example, in the pancreas. To verify the presence of this capability in zsig58 polypeptides, agonists or antagonists of the present invention, such zsig58 polypeptides, agonists or antagonists are evaluated with respect to their ability to facilitate wound healing according to procedures known in the art. If desired, zsig58 polypeptide performance in this regard can be compared to growth factors, such as EGF, NGF, TGF-α, TGF-β, insulin, IGF-I, IGF-II, fibroblast growth factor (FGF) and the like. In addition, zsig58 polypeptides or agonists or antagonists thereof may be evaluated in combination with one or more growth factors to identify synergistic effects.

In addition, zsig58 polypeptides, agonists or antagonists thereof may be therapeutically useful for anti-microbial applications. To verify the presence of this capability in zsig58 polypeptides, agonists or antagonists of the present invention, such zsig58 polypeptides, agonists or antagonists are evaluated with respect to their antimicrobial properties according to procedures known in the art. See, for example, Barsum et al., Eur. Respir. J. 8(5): 709-14, 1995; Sandovsky-Losica et al., J. Med. Vet. Mycol (England) 28(4): 279-87, 1990; Mehentee et al., J. Gen. Microbiol (England) 135 (Pt. 8): 2181-8, 1989; Segal and Savage, Journal of Medical and Veterinary Mycology 24: 477-479, 1986 and the like. If desired, zsig58 polypeptide performance in this regard can be compared to proteins known to be functional in this regard, such as proline-rich proteins, lysozyme, histatins, lactoperoxidase or the like. In addition, zsig58 polypeptides or agonists or antagonists thereof may be evaluated in combination with one or more antimicrobial agents to identify synergistic effects.

Anti-microbial protective agents may be directly acting or indirectly acting. Such agents operating via membrane association or pore forming mechanisms of action directly attach to the offending microbe. Anti-microbial agents can also act via an enzymatic mechanism, breaking down microbial protective substances or the cell wall/membrane thereof. Anti-microbial agents, capable of inhibiting microorganism proliferation or action or of disrupting microorganism integrity by either mechanism set forth above, are useful in methods for preventing contamination in cell culture by microbes susceptible to that anti-microbial activity. Such techniques involve culturing cells in the- presence of an effective amount of said zsig58 polypeptide, or an agonist or antagonist thereof.

Also, zsig58 polypeptides or agonists thereof may be used as cell culture reagents in in vitro studies of exogenous microorganism infection, such as bacterial, viral or fungal infection. Such moieties may also be used in in vivo animal models of infection. Also, the microorganism-adherence properties of zsig58 polypeptides or agonists thereof can be studied under a variety of conditions in binding assays and the like.

Bone cell precursors, such as osteoblasts and osteoclasts, are generated from bone marrow, but are greatly influenced by the hormones produced in the ovaries. Given the ovarian localization of the present invention, assays that measure bone formation and/or resorption are important assays to assess zsig58 activity. One example is an assay system that permits rapid identification of substances having selective calcitonin receptor activity on cells expressing the calcitonin receptor. The calcitonin receptor is a member of the G-protein receptor family and transduces signal via activation of adenylate cyclase, leading to elevation of cellular cAMP levels (Lin et al., Science 254:1022-24, 1991). This assay system exploits the receptor's ability to elevate cAMP levels as a way to detect other molecules that are able to stimulate the calcitonin receptor and initiate signal transduction.

Receptor activation can be detected by: (1) measurement of adenylate cyclase activity (Salomon et al., Anal. Biochem. 58:541-48, 1974; Alvarez and Daniels, Anal. Biochem. 187:98-103, 1990); (2) measurement of change in intracellular cAMP levels using conventional radioimmunoassay methods (Steiner et al., J. Biol. Chem. 247:1106-13, 1972; Harper and Brooker, J. Cyc. Nucl. Res. 1:207-18, 1975); or (3) through use of a cAMP scintillation proximity assay (SPA) method (Amersham Corp., Arlington Heights, Ill.).

An alternative assay system involves selection of polypeptides that are able to induce expression of a cyclic AMP response element (CRE)-luciferase reporter gene, as a consequence of elevated cAMP levels, in cells expressing a calcitonin receptor, but not in cells lacking calcitonin receptor expression, as described in U.S. Pat. Nos. 5,622,839, 5,674,689, and U.S. Pat. No. 5,674,981.

Well established animal models are available to test in vivo efficacy of zsig58 polypeptides that interact with the calcitonin receptor. Moreover, these models may be used to test effects of zsig58 on bone other than through the calcitonin receptor. For example, the hypocalcemic rat or mouse model can be used to determine the effect on serum calcium, and the ovariectomized rat or mouse can be used as a model system for osteoporosis. Bone changes seen in these models and in humans during the early stages of estrogen deficiency are qualitatively similar. Calcitonin has been shown to be an effective agent for the prevention of bone loss in ovariectomized women and rats (Mazzuoli et al., Calcif. Tissue Int. 47:209-14, 1990; Wronski et al., Endocrinology 129:2246-50, 1991). High dose estrogen has been shown to inhibit bone resorption and to stimulate bone formation in an ovariectomized mouse model (Bain et al., J. Bone Miner. Res. 8:435-42, 1993).

Biologically active zsig58 polypeptides of the present invention that interact with the calcitonin receptor, hormonal production from the ovaries, or exert other effects on bone, are therefore contemplated to be advantageous for use in therapeutic applications for which calcitonin is useful. Such applications, for example, are in the treatment of osteoporosis, Paget's disease, hyperparathyroidism, osteomalacia, idiopathic hypercalcemia of infancy and other conditions. Additional applications are to inhibit gastric secretion in the treatment of acute pancreatitis and gastrointestinal disorders, and uses as analgesics, in particular for bone pain.

In vivo assays for measuring changes in bone formation rates include performing bone histology (see, Recker, R., eds. Bone Histomorphometry: Techniques and Interpretation. Boca Raton: CRC Press, Inc., 1983) and quantitative computed tomography (QCT; Ferretti,J. Bone 17:353S-364S, 1995; Orphanoludakis et al., Investig. Radiol. 14:122-130, 1979; and Durand et al., Medical Physics 19:569-573, 1992). An exemplary ex vivo assay for measuring changes in bone formation is a calavarial assay (Gowen et al., J. Immunol. 136:2478-2482, 1986) or resorption calvarial assay (Linkhart, T. A., and Mohan, S., Endocrinology 125:1484-1491, 1989).

Proteins of the present invention are useful for example, in treating ovarian, pancreatic, ocular, blood or bone disorders, can be measured in vitro using cultured cells or in vivo by administering molecules of the claimed invention to the appropriate animal model. For instance, host cells expressing a secreted form of zsig58 polypeptide may be embedded in an alginate environment and injected (implanted) into recipient animals. Alginate-poly-L-lysine microencapsulation, permselective membrane encapsulation and diffusion chambers are a means to entrap transfected mammalian cells or primary mammalian cells to permit the diffusion of proteins and other macromolecules secreted or released by the captured cells to the recipient animal. Most importantly, the capsules mask and shield the foreign, embedded cells from the recipient animal's immune response. Such encapsulations can extend the life of the injected cells from a few hours or days (naked cells) to several weeks (embedded cells). Alginate threads provide a simple and quick means for generating embedded cells and testing, in vivo, the proteins secreted therefrom.

The materials needed to generate the alginate threads are known in the art. In an exemplary procedure, 3% alginate is prepared in sterile H₂O, and sterile filtered. Just prior to preparation of alginate threads, the alginate solution is again filtered. An approximately 50% cell suspension (containing about 5×10⁵ to about 5×10⁷ cells/ml) is mixed with the 3% alginate solution. One ml of the alginate/cell suspension is extruded into a 100 mM sterile filtered CaCl₂ solution over a time period of ˜15 min, forming a “thread”. The extruded thread is then transferred into a solution of 50 mM CaCl₂, and then into a solution of 25 mM CaCl₂. The thread is then rinsed with deionized water before coating the thread by incubating in a 0.0% solution of poly-L-lysine. Finally, the thread is rinsed with Lactated Ringer's Solution and drawn from solution into a syringe barrel (without needle). A large bore needle is then attached to the syringe, and the thread is intraperitoneally injected into a recipient in a minimal volume of the Lactated Ringer's Solution.

An alternative in vivo approach for assaying proteins of the present invention involves viral delivery systems. Exemplary viruses for this purpose include adenovirus, herpesvirus, retroviruses, vaccinia virus, and adeno-associated virus (AAV). Adenovirus, a double-stranded DNA virus, is currently the best studied gene transfer vector for delivery of heterologous nucleic acid (see T. C. Becker et al., Meth. Cell Biol. 43:161-89, 1994; and J. T. Douglas and D. T. Curiel, Science & Medicine 4:44-53, 1997). The adenovirus system offers several advantages: (i) adenovirus can accommodate relatively large DNA inserts; (ii) can be grown to high-titer; (iii) infect a broad range of mammalian cell types; and (iv) can be used with many different promoters including ubiquitous, tissue specific, and regulatable promoters. Also, because adenoviruses are stable in the bloodstream, they can be administered by intravenous injection.

Using adenovirus vectors where portions of the adenovirus genome are deleted, inserts are incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an El gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are El deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293 cells can be grown as adherent cells or in suspension culture at relatively high cell density to produce significant amounts of protein (See Garnier et al., Cytotechnol. 15:145-55, 1994). With either protocol, an expressed, secreted heterologous protein can be repeatedly isolated from the cell culture supernatant, lysate, or membrane fractions depending on the disposition of the expressed protein in the cell. Within the infected 293 cell production protocol, non-secreted proteins may also be effectively obtained.

As a ligand, the activity of zsig58 polypeptide can be measured by a silicon-based biosensor microphysiometer which measures the extracellular acidification rate or proton excretion associated with receptor binding and subsequent physiologic cellular responses. An exemplary device is the Cytosensor™ Microphysiometer manufactured by Molecular Devices, Sunnyvale, CA. A variety of cellular responses, such as cell proliferation, ion transport, energy production, inflammatory response, regulatory and receptor activation, and the like, can be measured by this method. See, for example, McConnell, H. M. et al., Science 257:1906-1912, 1992; Pitchford, S. et al., Meth. Enzymol. 228:84-108, 1997; Arimilli, S. et al., J. Immunol. Meth. 212:49-59, 1998; Van Liefde, I. et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including zsig58 polypeptide, its agonists, or antagonists. Preferably, the microphysiometer is used to measure responses of a zsig58-responsive eukaryotic cell, compared to a control eukaryotic cell that does not respond to zsig58 polypeptide. Zsig58-responsive eukaryotic cells comprise cells into which a receptor for zsig58 has been transfected creating a cell that is responsive to zsig58; or cells naturally responsive to zsig58 such as cells derived from ovarian and pancreatic tissue. Differences, measured by a change, for example, an increase or diminution in extracellular acidification, in the response of cells exposed to zsig58 polypeptide, relative to a control not exposed to zsig58, are a direct measurement of zsig58-modulated cellular responses. Moreover, such zsig58-modulated responses can be assayed under a variety of stimuli. Using the microphysiometer, there is provided a method of identifying agonists of zsig58 polypeptide, comprising providing cells responsive to a zsig58 polypeptide, culturing a first portion of the cells in the absence of a test compound, culturing a second portion of the cells in the presence of a test compound, and detecting a change, for example, an increase or diminution, in a cellular response of the second portion of the cells as compared to the first portion of the cells. The change in cellular response is shown as a measurable change extracellular acidification rate. Moreover, culturing a third portion of the cells in the presence of zsig58 polypeptide and the absence of a test compound can be used as a positive control for the zsig58-responsive cells, and as a control to compare the agonist activity of a test compound with that of the zsig58 polypeptide. Moreover, using the microphysiometer, there is provided a method of identifying antagonists of zsig58 polypeptide, comprising providing cells responsive to a zsig58 polypeptide, culturing a first portion of the cells in the presence of zsig58 and the absence of a test compound, culturing a second portion of the cells in the presence of zsig58 and the presence of a test compound, and detecting a change, for example, an increase or a diminution in a cellular response of the second portion of the cells as compared to the first portion of the cells. The change in cellular response is shown as a measurable change extracellular acidification rate. Antagonists and agonists, for zsig58 polypeptide, can be rapidly identified using this method.

Moreover, zsig58 can be used to identify cells, tissues, or cell lines which respond to a zsig58-stimulated pathway. The microphysiometer, described above, can be used to rapidly identify ligand-responsive cells, such as cells responsive to zsig58 of the present invention. Cells can be cultured in the presence or absence of zsig58 polypeptide. Those cells which elicit a measurable change in extracellular acidification in the presence of zsig5B are responsive to zsig58. Such cell lines, can be used to identify antagonists and agonists of zsig58 polypeptide as described above.

In view of the tissue distribution observed for zsig58, agonists (including the natural ligand/ substrate/ cofactor/ etc.) and antagonists have enormous potential in both in vitro and in vivo applications. Compounds identified as zsig58 agonists are useful for stimulating cell growth or signal transduction in vitro and in vivo. For example, zsig58 and agonist compounds are useful as components of defined cell culture media, and may be used alone or in combination with other cytokines and hormones to replace serum that is commonly used in cell culture. Agonists are thus useful in specifically promoting the growth and/or development of cells in culture. Considering the high expression of zsig58 ovary and pancreas, zsig58 polypeptides and zsig58 agonists may be particularly useful as research reagents, particularly for the growth of pancreatic cell types and ovarian cell lines, human eggs, cells from animal embryos or primary cultures derived from these tissues. As such, zsig58 polypeptide can be provided as a supplement in cell culture medium.

Antagonists are also useful as research reagents for characterizing sites of ligand-receptor interaction. Inhibitors of zsig58 activity (zsig58 antagonists) include anti-zsig58 antibodies and soluble proteins which bind zsig58 polypeptide, as well as other peptidic and non-peptidic agents (including ribozymes).

Zsig58 polypeptide can be used to identify inhibitors (antagonists) of its activity. Test compounds are added to the biological or biochemical assays disclosed herein to identify compounds that inhibit the activity of zsig58. In addition to those assays disclosed herein, samples can be tested for inhibition of zsig58 activity within a variety of assays designed to measure receptor binding or the stimulation/inhibition of zsig58-dependent cellular responses. For example, zsig58-responsive cell lines can be transfected with a reporter gene construct that is responsive to a zsig58-stimulated cellular pathway. Reporter gene constructs of this type are known in the art, and will generally comprise a zsig58-DNA response element operably linked to a gene encoding an assay detectable protein, such as luciferase. DNA response elements can include, but are not limited to, cyclic AMP response elements (CRE), hormone response elements (HRE) insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6; 1988 and Habener, Molec. Endocrinol. 4 (8):1087-94; 1990. Hormone response elements are reviewed in Beato, Cell 56:335-44; 1989. Candidate compounds that serve as test samples including solutions, mixtures or extracts, are tested for the level of response to the zsig58 polypeptide. The ability of a test sample to inhibit the activity of zsig58 polypeptide on the target cells as evidenced by a decrease in zsig58 stimulation of reporter gene expression in the presence of a test sample relative to a control which was cultured in the absence of a test sample. Assays of this type will detect compounds that directly block zsig58 binding to cell-surface receptors, e.g., dimerization, as well as compounds that block processes in the cellular pathway subsequent to receptor-ligand binding. Alternatively, compounds or other samples can be tested for direct blocking of zsig58 binding to receptor using zsig58 tagged with a detectable label (e.g., ¹²⁵I, biotin, horseradish peroxidase, FITC, and the like). Within assays of this type, the ability of a test sample to inhibit the binding of labeled zsig58 to the receptor is indicative of inhibitory activity, which can be confirmed through secondary assays. Receptors used within binding assays may be cellular receptors or isolated, immobilized receptors.

Alternatively, the above methodology may be used to identify agonists of zsig58 activity. Candidate compounds serving as test samples including solutions, mixtures or extracts, are tested for the ability to mimic the activity of zsig58 polypeptide on the target cells as evidenced by stimulation of reporter gene expression in the presence of a test sample and the absence of zsig58, relative to a control (cultured in the absence of a test sample and the absence of zsig58 polypeptide), using assays as described above.

A zsig58 polypeptide can be expressed as a fusion with an immunoglobulin heavy chain constant region, typically an F_{C} fragment, which contains two constant region domains and lacks the variable region. Methods for preparing such fusions are disclosed in U.S. Pat. Nos. 5,155,027 and 5,567,584. Such fusions are typically secreted as multimeric molecules wherein the Fc portions are disulfide bonded to each other and two non-Ig olypeptides are arrayed in closed proximity to each other. Fusions of this type can be used to affinity purify ligand, as an in vitro assay tool, or an antagonist of zsig58. For use in assays, the chimeras are bound to a support via the Fc region and used in an ELISA format.

A zsig58 polypeptide can also be used for purification of receptor or polypeptides to which it binds. The zsig58 polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and membrane fractions containing receptors are passed through the column one or more times to allow the receptor to bind to the ligand zsig58 polypeptide. The receptor is then eluted using changes in salt concentration, chaotropic agents (guanidine HCl), or pH to disrupt ligand-receptor binding.

An assay system that uses a ligand-binding receptor (or an antibody, one member of a complement/anti-complement pair) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

Ligand-binding receptor polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

Zsig58 polypeptides can also-be used to prepare antibodies that bind to zsig58 epitopes, peptides or polypeptides. The zsig58 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a zsig58 polypeptide (e.g., SEQ ID NO:2). Polypeptides comprising a larger portion of a zsig58 polypeptide, i.e., from 30 to 10 residues up to the entire length of the amino acid sequence are included. Antigens or immunogenic epitopes can also include attached tags, adjuvants and carriers, as described herein. Suitable antigens include the zsig58 polypeptide encoded by SEQ ID NO:2 from amino acid number 26(Thr) to amino acid number 402 (Lys), or a contiguous 9 to 402 AA amino acid fragment thereof. Preferred peptides to use as antigens are hydrophilic peptides such as those predicted by one of skill in the art from a hydrophobicity plot, for example, from a Hopp/Woods hydrophilicity profile based on a sliding six-residue window, with buried G, S, and T residues and exposed H, Y, and W residues ignored. In addition, the carboxy domain, conserved motifs, variable regions between conserved motifs of zsig58, and the flanking region N-terminal to the carboxy domain, described herein, are suitable. Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982. Specifically binding anti-zsig58 antibodies can be detected by a number of methods in the art, and disclosed below.

As would be evident to one of ordinary skill in the art, polyclonal antibodies can be generated from inoculating a variety of warm-blooded animals such as horses, cows, goats, sheep, dogs, chickens, rabbits, mice, and rats with a zsig58 polypeptide or a fragment thereof. The immunogenicity of a zsig58 polypeptide may be increased through the use of an adjuvant, such as alum (aluminum hydroxide) or Freund's complete or incomplete adjuvant. Polypeptides useful for immunization also include fusion polypeptides, such as fusions of zsig58 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Moreover, human antibodies can be produced in transgenic, non-human animals that have been engineered to contain human immunoglobulin genes as disclosed in WIPO Publication WO 98/24893. It is preferred that the endogenous immunoglobulin genes in these animals be inactivated or eliminated, such as by homologous recombination.

Antibodies are considered to be specifically binding if: 1) they exhibit a threshold level of binding activity, and 2) they do not significantly cross-react with related polypeptide molecules. A threshold level of binding is determined if anti-zsig58 antibodies herein bind to a zsig58 polypeptide, peptide or epitope with an affinity at least 10-fold greater than the binding affinity to control (non-zsig58) polypeptide. It is preferred that the antibodies exhibit a binding affinity (Kₐ) of 10⁶ M⁻¹ or greater, preferably 10 M or greater, more preferably 10⁸ M⁻¹ or greater, and most preferably 10⁻⁹ M⁻¹ or greater. The binding affinity of an antibody can be readily determined by one of ordinary skill in the art, for example, by Scatchard analysis (Scatchard, G., Ann. NY Acad. Sci. 51: 660-672, 1949).

Whether anti-zsig58 antibodies do not significantly cross-react with related polypeptide molecules is shown, for example, by the antibody detecting zsig58 polypeptide but not known related polypeptides using a standard Western blot analysis (Ausubel et al., ibid.). Examples of known related polypeptides are those disclosed in the prior art, such as known orthologs (e.g., PIR_{—}173639 (SEQ ID NO:3));and paralogs (e.g. TIGR (SEQ ID NO:4)) and similar known members of a protein family, Screening can also be done using non-human zsig58, and zsig58 mutant polypeptides. Moreover, antibodies can be “screened against” known related polypeptides to isolate a population that specifically binds to the zsig58 polypeptides. For example, antibodies raised to zsig58 are adsorbed to related polypeptides adhered to insoluble matrix; antibodies specific to zsig58 will flow through the matrix under the proper buffer conditions. Screening allows isolation of polyclonal and monoclonal antibodies non-crossreactive to known closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art. See, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67:101, 1984.

Alternative techniques for generating or selecting antibodies useful herein include in vitro exposure of lymphocytes to zsig58 protein or peptide, and selection of antibody display libraries in phage or similar vectors (for instance, through use of immobilized or labeled zsig58 protein or peptide). Genes encoding polypeptides having potential zsig58 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the zsig58 sequences disclosed herein to identify proteins which bind to zsig58. These “binding proteins” which interact with zsig58 polypeptides can be used for tagging cells; for isolating homolog polypeptides by affinity purification; they can be directly or indirectly conjugated to drugs, toxins, radionuclides and the like. These binding proteins can also be used in analytical methods such as for screening expression libraries and neutralizing activity. The binding proteins can also be used for diagnostic assays for determining circulating levels of polypeptides; for detecting or quantitating soluble polypeptides as marker of underlying pathology or disease. These binding proteins can also act as zsig58 “antagonists” to block zsig58 binding, zsig58-mediated cell-cell interactions, and signal transduction in vitro and in vivo.

A variety of assays known to those skilled in the art can be utilized to detect antibodies which specifically bind to zsig58 proteins or polypeptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant zsig58 protein or polypeptide.

Antibodies to zsig58 may be used for tagging cells that express zsig58; for isolating zsig58 by affinity purification; for diagnostic assays for determining circulating levels ozsig58 polypeptides; for detecting or quantitating soluble zsig58 as marker of underlying pathology or disease; in analytical methods employing FACS; for screening expression libraries; for generating anti-idiotypic antibodies; and as neutralizing antibodies or as antagonists to block zsig58 activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to zsig58 or fragments thereof may be used in vitro to detect denatured zsig58 or fragments thereof in assays, for example, Western Blots or other assays known in the art.

Antibodies, binding proteins or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, zsig58 polypeptides or anti-zsig58 antibodies, or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion. For these purposes, biotin/streptavidin is an exemplary complementary/anticomplementary pair.

In another embodiment, polypeptide-toxin fusion proteins or antibody-toxin fusion proteins can be used for targeted cell or tissue inhibition or ablation (for instance, to treat cancer cells or tissues). Alternatively, if the polypeptide has multiple functional domains (i.e., an activation domain or a ligand binding domain, plus a targeting domain), a fusion protein including only the targeting domain may be suitable for directing a detectable molecule, a cytotoxic molecule or a complementary molecule to a cell or tissue type of interest. In instances where the domain only fusion protein includes a complementary molecule, the anticomplementary molecule can be conjugated to a detectable or cytotoxic molecule. Such domain-complementary molecule fusion proteins thus represent a generic targeting vehicle for cell/tissue-specific delivery of generic anticomplementary-detectable/cytotoxic molecule conjugates.

In another embodiment, zsig58-cytokine fusion proteins or antibody-cytokine fusion proteins can be used for enhancing in vivo killing of target tissues (for example, ovarian and pancreatic cancers), if the zsig58 polypeptide or anti-zsig58 antibody targets, for example, the hyperproliferative blood or bone marrow cell (See, generally, Hornick et al., Blood 89:4437-47, 1997). They described fusion proteins enable targeting of a cytokine to a desired site of action, thereby providing an elevated local concentration of cytokine. Suitable zsig58 polypeptides or anti-zsig58 antibodies target an undesirable cell or tissue (i.e., a tumor or a leukemia), and the fused cytokine mediated improved target cell lysis by effector cells. Suitable cytokines for this purpose include interleukin 2 and granulocyte-macrophage colony-stimulating factor (GM-CSF), for instance.

In yet another embodiment, if the zsig58 polypeptide or anti- zsig58 antibody targets vascular cells or tissues, such polypeptide or antibody may be conjugated with a radionuclide, and particularly with a beta-emitting radionuclide, to reduce restenosis. Such therapeutic approach poses less danger to clinicians who administer the radioactive therapy. For instance, iridium-192 impregnated ribbons placed into stented vessels of patients until the required radiation dose was delivered showed decreased tissue growth in the vessel and greater luminal diameter than the control group, which received placebo ribbons. Further, revascularisation and stent thrombosis were significantly lower in the treatment group. Similar results are predicted with targeting of a bioactive conjugate containing a radionuclide, as described herein.

The bioactive polypeptide or antibody conjugates described herein can be delivered intravenously, intraarterially or intraductally, or may be introduced locally at the intended site of action.

Molecules of the present invention can be used to identify and isolate receptors to which zsig58 interacts or binds. For example, proteins and peptides of the present invention can be immobilized on a column and membrane preparations run over the column (Immobilized Affinity Ligand Technicues, Hermanson et al., eds., Academic Press, San Diego, Calif., 1992, pp.195-202). Proteins and peptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62 483-514, 1993 and Fedan et al., Biochem. Pharmacol. 33:1167-80, 1984) and specific cell-surface proteins can be identified.

The polypeptides, nucleic acid and/or antibodies of the present invention may be used in treatment of disorders associated with gonadal development, pregnancy, pubertal changes, menopause, ovarian cancer, fertility, ovarian function, polycystic ovarian syndrome, pancreas, diabetes, eye disease, pituitary function, osteoporosis, and other bone diseases. The molecules of the present invention may used to modulate or to treat or prevent development of pathological conditions in such diverse tissue as pancreas and ovary. In particular, certain syndromes or diseases may be amenable to such diagnosis, treatment or prevention. Moreover, natural functions, such as ovulation, may be suppressed or controlled for use in birth control by molecules of the present invention.

The polypeptides, nucleic acids and/or antibodies of the present invention may be used in treatment of disorders associated with diabetes. Molecules of the present invention may also be useful for revascularization in the eye, for complications related to poor circulation such as diabetic foot ulcers, for stroke, and other indications where angiogenesis is of benefit.

The zsig58 polypeptide is expressed in the pancreas. Thus, zsig58 polypeptide pharmaceutical compositions of the present invention may be useful in prevention or treatment of pancreatic disorders associated with pathological regulation of the expansion of neuroendocrine and exocrine cells in the pancreas, such as IDDM, pancreatic cancer, pathological regulation of blood glucose levels, insulin resistance or digestive function.

The zsig58 polypeptide of the present invention may act in the neuroendocrine/exocrine cell fate decision pathway and is therefore capable of regulating the expansion of neuroendocrine and exocrine cells in the pancreas. One such regulatory use is that of islet cell regeneration. Also, it has been hypothesized that the autoimmunity that triggers IDDM starts in utero, and zsig58 polypeptide is a developmental gene involved in cell partitioning. Assays and animal models are known in the art for monitoring the exocrine/neuroendocrine cell lineage decision, for observing pancreatic cell balance and for evaluating zsig58 polypeptide, fragment, fusion protein, antibody, agonist or antagonist in the prevention or treatment of the conditions set forth above.

Also, zsig58 polypeptide is expressed in the ovary and may have additional biological activity independent of pancreatic function, as described below.

Oogenesis is the process by which a diploid stem cell proceeds through multiple stages of differentiation, culminating in the formation of a terminally differentiated cell with a unique function, an oocyte. Unlike spermatogenesis, which begins at puberty and continues on through the life of a male, oogenesis begins during fetal development and by birth, a female's entire supply of primary oocytes are stored in the ovaries in primordial follicles and await maturation and release.

In the adult ovary, folliculogenesis starts when the follicles enter the growth phase. Early growing follicles undergo a dramatic process of cellular proliferation and differentiation. The classic control of ovarian function by luteinizing hormone (LH) and follicle stimulating hormone (FSH) is now thought to include the action of a variety of molecules that act to promote cell-cell interactions between cells of the follicle. For review, see Gougeon, A., Endocrine Rev. 17:121-155, 1996. Hence, the mechanisms for controlling ovarian folliculogenesis and dominant follicle selection are still under investigation. As zsig58 is expressed in the ovary, it may serve a role in regulating folliculogenesis and dominant follicle selection, by affecting proliferation or differentiation of follicular cells, affecting cell-cell interactions, modulating hormones involved in the process, and the like.

The ovarian cycle in mammals includes the growth and maturation of follicles, followed by ovulation and transformation of follicles into corpea lutea. The physiological events in the ovarian cycle are dependent on interactions between hormones and cells within the hypothalamic-pituitary-ovarian axis, including gonadotropin releasing hormone (GnRH), LH, and FSH. In addition, estradiol, synthesized in the follicle, primes the hypothalamic-pituitary axis and is required for the mid-cycle surge of gonadotropin that stimulates the resumption of oocyte meiosis and leads to ovulation and subsequent extrusion of an oocyte from the follicle. This gonadotropin surge also promotes the differentiation of the follicular cells from secreting estradiol to secreting progesterone. Progesterone, secreted by the corpus luteum, is needed for uterine development required for the implantation of fertilized oocytes. The central role of hyp)othalamic-pituitary-gonadal hormones in the ovarian cycle and reproductive cascade, and the role of sex steroids on target tissues and organs, e.g., uterus, breast, adipose, bones and liver, has made modulators of their activity desirable for therapeutic applications. Such applications include treatments for precocious puberty, endometriosis, uterine leiomyomata, hirsutism, infertility, pre menstrual syndrome (PMS), amenorrhea, and as contraceptive agents.

Zsig58 polypeptides, agonists and antagonists which modulate the actions of such hormones can be of therapeutic value. Such molecules can also be useful for modulating steroidogenesis, both in vivo and in vitro, and modulating aspects of the ovarian cycle such as oocyte maturation, ovarian cell-cell interactions, follicular development and rupture, luteal function, and promoting uterine implantation of fertilized oocytes. Molecules which modulate hormone action can be beneficial therapeutics for use prior to or at onset of puberty. For example, puberty in females is marked by an establishment of feed-back loops to control hormone levels and hormone production. Abnormalities resulting from hormone imbalances during puberty have been observed and include precocious puberty, where pubertal changes occur in females prior to the age of 8. Hormone-modulating molecules, can be used, in this case, to suppress hormone secretion and delay onset of puberty.

The level and ratio of gonadotropin and steroid hormones can be used to assess the existence of hormonal imbalances associated with diseases, as well as determine whether normal hormonal balance has been restored after administration of a therapeutic agent. Determination of estradiol, progesterone, LH, and FSH, for example, from serum is known by one of skill in the art. Such assays can be used to monitor the hormone levels after administration of zsig58 in vivo, or in a transgenic mouse model where the zsig58 gene is expressed or the murine ortholog is deleted. Thus, as a hormone-modulating molecule, zsig58 polypeptide can have therapeutic application for treating, for example, breakthrough menopausal bleeding, as part of a therapeutic regime for pregnancy support, or for treating symptoms associated with polycystic ovarian syndrome (PCOS), PMS and menopause. In addition, other in vivo rodent models are known in the art to assay effects of zsig58 polypeptide on, for example, polycystic ovarian syndrome (PCOS).

Proteins of the present invention may also be used in applications for enhancing fertilization during assisted reproduction in humans and in animals. Such assisted reproduction methods are known in the art and include artificial insemination, in vitro fertilization, embryo transfer, and gamete intrafallopian transfer. Such methods are useful for assisting those who may have physiological or metabolic disorders that prevent or impede natural conception. Such methods are also used in animal breeding programs, e.g., for livestock, racehorses, domestic and wild animals, and could be used as methods for the creation of transgenic animals. Zsig58 polypeptides could be used in the induction of ovulation, either independently or in conjunction with a regimen of gonadotropins or agents such as clomiphene citrate or bromocriptine (Speroff et al., Induction of ovulation, Clinical Gynecologic Endocrinology and Infertility, 5^{th} ed., Baltimore, Williams & Wilkins, 1994). As such, proteins of the present invention can be administered to the recipient prior to fertilization or combined with the sperm, an egg or an egg-sperm mixture prior to in vitro or in vivo fertilization. Such proteins can also be mixed with oocytes prior to cryopreservation to enhance viability of the preserved oocytes for use in assisted reproduction.

The zsig58 polypeptides, agonists and antagonists of the present invention may be directly used as or incorporated into therapies for treating reproductive disorders. Disorders such as luteal phase deficiency would benefit from such therapy (Soules, “Luteal phase deficiency: A subtle abnormality of ovulation” in, Infertility: Evaluation and Treatment, Keye et al., eds., Philadelphia, W B Saunders, 1995). Moreover, administration of gonadotropin-releasing hormone is shown to stimulate reproductive behavior (Riskin and Moss, Res. Bull. 11:481-5, 1983; Kadar et al., Physiol. Behav. 51:601-5, 1992 and Silver et al., J. Neruoendocrin. 4:207-10, 199; King and Millar, Cell. Mol. Neurobiol., 15:5-23, 1995). Given the high prevalence of sexual dysfunction and impotence in humans, molecules, such as zsig58, which may modulate or enhance gonadotropin activity can find application in developing treatments for these conditions.

The zsig58 polypeptides of the present invention can be used to study ovarian cell proliferation, maturation, and differentiation, i.e., by acting as a luteinizing agent that converts granulosa cells from estradiol to progesterone-producing cells. Such methods of the present invention generally comprise incubating granulosa cells, theca cells, oocytes or a combination thereof, in the presence and absence of zsig58 polypeptide, monoclonal antibody, agonist or antagonist thereof and observing changes in cell proliferation, maturation and differentiation. See for example, Basini et al., (J. Rep. Immunol. 37:139-53, 1998); Duleba et al.,(Fert. Ster. 69:335-40, 1998); and Campbell, B. K. et al., J. Reprod. and Fert. 112:69-77, 1998).

The molecules of the present invention are useful as components of defined cell culture media, as described herein, and may be used alone or in combination with other cytokines and hormones to replace serum that is commonly used in cell culture. Molecules of the present invention are particularly useful in specifically promoting the growth, development, differentiation, and/or maturation of ovarian cells in culture, and may also prove useful in the study of the ovarian cycle, reproductive function, ovarian cell-cell interactions, and fertilization.

In addition, the present invention also provides methods for studying steroidogenesis and steroid hormone secretion. Such methods generally comprise incubating ovarian cells in culture medium comprising zsig58 polypeptides, monoclonal antibodies, agonists or antagonists thereof with and without gonadotropins and/or steroid hormones, and subsequently observing protein and steroid secretion. Exemplary gonadotropin hormones include luteinizing hormone and follicle stimulating hormone (Rouillier et al., Mol. Reprod. Dev. 50:170-7, 1998). Exemplary steroid hormones include estradiol, androstenedione, and progesterone. Effects of zsig58 on steroidogenesis or steroid secretion can be determined by methods known in the art, such as radioimmunoassay (to detect levels of estradiol, androstenedione, progesterone, and the like), and immunoradiometric assay (IRMA).

Molecules expressed in the ovaries, such as zsig58 polypeptide, and which may modulate hormones, hormone receptors, growth factors, or cell-cell interactions, of the reproductive cascade or are involved in oocyte or ovarian development, would be useful as markers for cancer of reproductive organs and as therapeutic agents for hormone-dependent cancers, by inhibiting hormone-dependent growth and/or development of tumor cells. Of all reproductive system cancers, ovarian cancer causes the most morbidity, and the number of cases has increased 30% over the last decade. Moreover, receptors for steroid hormones involved in the reproductive cascade are found in human tumors and tumor cell lines (breast, prostate, endometrial, ovarian, kidney, and pancreatic tumors) (Kakar et al., Mol. Cell. Endocrinol., 106:145-49, 1994; Kakar and Jennes, Cancer Letts., 98:57-62, 1995). Thus, expression of zsig58 in ovaries suggests that polypeptides of the present invention would be useful in diagnostic methods for the detection and monitoring of ovarian cancer.

Diagnostic methods of the present invention involve the detection of zsig58 polypeptides in the serum or tissue biopsy of a patient undergoing analysis of reproductive function or evaluation for possible ovarian cancer. Such polypeptides can be detected using immunoassay techniques and antibodies, described herein, that are capable of recognizing polypeptide epitopes. More specifically, the present invention contemplates methods for detecting zsig58 polypeptides comprising:

exposing a test sample potentially containing zsig58 polypeptides to an antibody attached to a solid support, wherein said antibody binds to a first epitope of a zsig58 polypeptide;

washing the immobilized antibody-polypeptide to remove unbound contaminants;

exposing the immobilized antibody-polypeptide to a second antibody directed to a second epitope of a zsig58 polypeptide, wherein the second antibody is associated with a detectable label; and

detecting the detectable label. Altered levels of zsig58 polypeptides in a test sample, such as serum sweat, saliva, biopsy, and the like, can be monitored as an indication of reproductive function or of ovarian cancer or disease, when compared against a normal control.

Additional methods using probes or primers derived, for example, from the nucleotide sequences disclosed herein can also be used to detect zsig58 expression in a patient sample, such as a blood, saliva, sweat, tissue sample, or the like. For example, probes can be hybridized to tumor tissues and the hybridized complex detected by in situ hybridization. Zsig58 sequences can also be detected by PCR amplification using cDNA generated by reverse translation of sample mRNA as a template (PCR Primer A Laboratorv Manual, Dieffenbach and Dveksler, eds., Cold Spring Harbor Press, 1995). When compared with a normal control, both increases or decreases of zsig58 expression in a patient sample, relative to that of a control, can be monitored and used as an indicator or diagnostic for disease.

Polynucleotides encoding zsig58 polypeptides are useful within gene therapy applications where it is desired to increase or inhibit zsig58 activity. If a mammal has a mutated or absent zsig58 gene, the zsig58 gene can be introduced into the cells of the mammal. In one embodiment, a gene encoding a zsig58 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. Such a defective virus is not infective after introduction into a cell. Use of defective viral vectors allows for administration to cells in a specific, localized area, without concern that virus will be produced and the vector transferred to other cells via infection. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a zsig58 gene can be introduced in a retroviral vector, e.g., as described in Anderson et al., U.S. Pat. No. 5,399,346; Mann et al., Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Patent Publication No. WO 95/07358, published Mar. 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, ovary, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

It is possible to remove the target cells from the body; to introduce the vector as a naked DNA plasmid; and then to re-implant the transformed cells into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Antisense methodology can be used to inhibit zsig58 gene transcription, such as to inhibit cell proliferation in vivo. Polynucleotides that are complementary to a segment of a zsig58-encoding polynucleotide (e.g., a polynucleotide as set froth in SEQ ID NO:1) are designed to bind to zsig58-encoding MRNA and to inhibit translation of such MRNA. Such antisense polynucleotides are used to inhibit expression of zsig58 polypeptide-encoding genes in cell culture or in a subject.

The present invention also provides reagents which will find use in diagnostic applications. For example, the zsig58 gene, a probe comprising zsig58 DNA or RNA or a subsequence thereof can be used to determine if the zsig58 gene is present on chromosome 11 or if a mutation has occurred. Zsig58 is located at the 11p15.4-p15.3 region of chromosome 11 (see, Example 3). Detectable chromosomal aberrations at the zsig58 gene locus include, but are not limited to, aneuploidy, gene copy number changes, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995).

The precise knowledge of a gene's position can be useful for a number of purposes, including: 1) determining if a sequence is part of an existing contig and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which may aid in determining what function a particular gene might have.

The zsig58 gene is located at the 11p15.4-p15.3 region of chromosome 11. Several genes of known function map to this region. Parathyroid hormone maps to 11p15.3-p15.1 and is associated with controlling calcium balance in the blood. See, for example, Phillips et al., Am. J. Hum. Genet. 59: 613-619, 1996. Calcitonin/calcitonin-related peptide alpha and beta, a peptide hormone that reduces serum calcium maps to chromosome 11p15.2-p15.1. See for example, Hoovers et al., Genomics 15:525-9, 1993. The sulfonylurea receptor found on pancreatic cells and associated with promotion of insulin secretion maps to chromosome 11p15.1. See, for example, Thomas et al., Science 268:426-9, 1995. Inwardly-rectifying potassium channel, found in cardiac muscle, pancreatic beta cells, pituitary tissue, skeletal muscle, brain and muscle and are crucial regulators of glucose-induced insulin secretion map to chromosome 11p15.1. See for example, Inagaki et al., Neuron 16:1011-17, 1996 A breast cancer tumor susceptibility gene maps to 11p15.2-p15.l. See for example, Li et al., Cell 88:143-54, 1997. Cardiac LIM protein, a positive regulator of myogenesis, maps. to chromosome 11p15.1. See for example, Fung et al., Genomics 28:602-3, 1995. All of these serve as possible candidate genes for an inheritable disease which show linkage to the same chromosomal region as the zsig58 gene.

Mice engineered to express the zsig58 gene, referred to as “transgenic mice,” and mice that exhibit a complete absence of zsig58 gene function, referred to as “knockout mice,” may also be generated (Snouwaert et al., Science 257:1083, 1992; Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express zsig58, either ubiquitously or under a tissue-specific or tissue-restricted promoter can be used to ask whether over-expression causes a phenotype. For example, over-expression of a wild-type zsig58 polypeptide, polypeptide fragment or a mutant thereof may alter normal cellular processes, resulting in a phenotype that identifies a tissue in which zsig58 expression is functionally relevant and may indicate a therapeutic target for the zsig58, its agonists or antagonists. For example, a preferred transgenic mouse to engineer is one that over-expresses the zsig58 mature polypeptide (approximately residue 26 (Thr) to residue 402 (Lys) of SEQ ID NO:2). Moreover, such over-expression may result in a phenotype that shows similarity with human diseases. Similarly, knockout zsig58 mice can be used to determine where zsig58 is absolutely required in vivo. The phenotype of knockout mice is predictive of the in vivo effects of that a zsig58 antagonist, such as those described herein, may have. The human zsig58 cDNA can be used to isolate murine zsig58 mRNA, cDNA and genomic DNA, which are subsequently used to generate knockout mice. These mice may be employed to study the zsig58 gene and the protein encoded thereby in an in vivo system, and can be used as in vivo models for corresponding human diseases. Moreover, transgenic mice expression of zsig58 antisense polynucleotides or ribozymes directed against zsig58, described herein, can be used analogously to transgenic mice described above. For pharmaceutical use, the proteins of the present invention are formulated for parenteral, particularly intravenous or subcutaneous, delivery according to conventional methods. Intravenous administration will be by bolus injection or infusion over a typical period of one to several hours. In general, pharmaceutical formulations will include a zsig58 protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Therapeutic doses will generally be determined by the clinician according to accepted standards, taking into account the nature and severity of the condition to be treated, patient traits, and the like. Determination of dose and duration of treatment is within the level of ordinary skill in the art. The invention is further illustrated by the following non-limiting examples.

### EXAMPLES

### Example 1

### Identification of zsig58

A. Using an EST Sequence to Obtain Full-length zsig58

Scanning of translated pancreas, liver, lung and breast library DNA databases using a signal trap as a query resulted in identification of an expressed sequence tag (EST) sequence found to be homologous to a human secretory signal sequence.

Confirmation of the EST sequence was made by sequence analyses of the cDNA from which the EST originated. This cDNA was contained in a plasmid from a human pituitary library, and was sequenced using the following primers to generate complete double stranded sequence of this clone: ZC976 (SEQ ID NO:7), ZC6768 (SEQ ID NO:8), ZC17601 (SEQ ID NO:9), ZC17602 (SEQ ID NO:10), ZC17766 (SEQ ID NO:11), ZC17767 (SEQ ID NO:12), ZC17676 (SEQ ID NO:13), ZC17677 (SEQ ID NO:14), ZC17852 (SEQ ID NO:15), and ZC17854 (SEQ ID NO:16). Analysis of the double stranded sequence of this plasmid insert revealed it was full length.

### Example 2

### Tissue Distribution

Northern blot analysis was performed using Human Multiple Tissue Blots (MTN I, MTN II, and MTN III) (Clontech). An insert from the full length clone, described in Example 1, was excised using EcoRI and XhoI (Boehringer) and gel purified using a commercially available kit (QiaexII™; Qiagen) and then radioactively labeled with ³²P-dCTP using Rediprime™ (Amersham), a random prime labeling system, according to the manufacturer's specifications. The probe was then purified using a Nuc-Trap™ column (Stratagene) according to the manufacturer's instructions. ExpressHybT™ (Clontech) solution was used for prehybridization and as a hybridizing solution for the Northern blots. Hybridization took place overnight at 55° C. using 3×10⁶ cpm/ml of labeled probe. The blots were then washed in 2× SSC/1% SDS at 65° C., followed by a wash in 0.1× SSC/0.1% SDS at 55° C. A transcript was detected. at approximately 3 kb with strong signals in ovary and pancreas. Weak signals were seen in small intestine, heart, spleen, colon, stomach, thyroid, spinal cord, lymph node, trachea and adrenal gland. No signals were apparent in other tissues represented on the blots.

Dot Blots were also performed using Human RNA Master BlotS™ (Clontech). The methods and conditions for the Dot Blots are the same as for the Multiple Tissue Blots disclosed above. Strong signal intensity was present in ovary, pancreas and small intestine. A moderate signal was present in many other tissues, including heart, aorta, colon, bladder, uterus, prostate, stomach, spleen, mammary gland, liver, appendix, lung, placenta, trachea, fetal heart, fetal kidney, fetal spleen and fetal lung.

### Example 3

### Chromosomal Assignment and Placement of Zsig58

Zsig58 was mapped to chromosome 11 using the commercially available version of the “Stanford G3 Radiation Hybrid Mapping Panel” (Research Genetics, Inc., Huntsville, Ala.). The “Stanford G3 RH Panel” contains DNAs from each of 83 radiation hybrid clones of the whole human genome, plus two control DNAs (the RM donor and the A3 recipient). A publicly available WWW server (http://shgc-www.stanford.edu) allows chromosomal localization of markers. For the mapping of Zsig58 with the “Stanford G3 RH Panel”, 20 μl reactions were set up in a PCRable 96-well microtiter plate (Stratagene, La Jolla, Calif.) and used in a “RoboCycler Gradient 96” thermal cycler (Stratagene). Each of the 85 PCR reactions consisted of 2 μl 10× KlenTaq PCR reaction buffer (Clontech Laboratories, Inc., Palo Alto, Calif.), 1.6 μl dNTPs mix (2.5 mM each, PERKIN-ELMER,Foster City, Calif.), 1 μl sense primer, ZC 18,333 (SEQ ID NO:17), 1 μl antisense primer, ZC 18,334 (SEQ ID NO):18), 2 μl “RediLoad” (Research Genetics), 0.4 μl 50× Advantage KlenTaq Polymerase Mix (Clontech), 25 ng of DNA from an individual hybrid clone or control and ddH₂O for a total volume of 20 pl. The reactions were overlaid with an equal amount of mineral oil and sealed. The PCR cycler conditions were as follows: an initial 1 cycle 5 minute denaturation at 95° C.; 35 cycles of a 1 minute denaturation at 95° C., 1 minute annealing at 62° C. and 1.5 minute extension at 72° C., followed by a final 1 cycle extension of 7 minutes at 72° C. The reactions were separated by electrophoresis on a 2% agarose gel (Life Technologies).

The results showed linkage of Zsig58 to the framework maker SHGC-30914 with a LOD score of >13 and at a distance of 10.40 cR_{—}10000 from the marker. The use of surrounding markers positions zsig58 in the 11p15.4-pl5.3 region on the integrated LDB chromosome 11 map (The Genetic Location Database, University of Southhampton, WWW server: http://cedar.genetics. soton.ac.uk/public_html/).

### Example 4

### Generation of Untagged zsig58 Recombinant Adenovirus

The protein coding region of human zsig58 was amplified by PCR using primers that added FseI and AscI restriction sties at the 5′ and 3′ termini respectively. PCR primers ZC18843 (SEQ ID NO:19) and ZC18844 (SEQ ID NO:20) were used with a template pBluescript (Stratagene) vector containing the full-length zsig58 cDNA (Example 1) in a PCR reaction as follows: one cycle at 95° C. for 5 minutes; followed by 15 cycles at 95° C. for 1 min., 61° C. for 1 min., and 72° C. for 1.5 min.; followed by 72° C. for 7 min.; followed by a 4° C. soak. The PCR reaction product was loaded onto a 1.2% (low melt) SeaPlaque GTG (FMC, Rockland, Me.) gel in TAE buffer. The zsig58 PCR product was excised from the gel and purified using the QIAquick™ PCR Purification Kit gel cleanup kit as per kit instructions (Qiagen). The PCR product was then digested with PmeI-AscI, phenol/chloroform extracted, EtOH precipitated, and rehydrated in 20 ml TE (Tris/EDTA pH 8). The 1209 bp zsig58 fragment was then ligated into the FseI-AscI sites of the transgenic vector pTG12-8 (See, example 5, below) and transformed into DH10B competent cells by electroporation. Clones containing zsig58 were identified by plasmid DNA miniprep followed by digestion with FseI-AscI. A positive clone was sent to the sequencing department to insure there are no deletions or other anomalies in the construct. The sequence of zsig58 cDNA was confirmed. Qiagen Maxi Prep protocol (Qiagen) was used to generate DNA to continue our process described below.

A. Preparation of DNA construct for generation of Adenovirus

The 1209 bp zsig58 cDNA was released from the TG12-8 vector using FseI and AscI enzymes. The cDNA was isolated on a 1% low melt SeaPlaque GTG™ (FMC, Rockland, Me.) gel and was then excised from the gel and the gel slice melted at 70° C., extracted twice with an equal volume of Tris buffered phenol, and EtOH precipitated. The DNA was resuspended in 10 μl H₂.O

The zsig58 cDNA was cloned into the FseI-AscI sites of a modified pAdTrack CMV (He, T-C. et al., PNAS 95:2509-2514, 1998). This construct contains the GFP marker gene. The CMV promoter driving GFP expression was replace with the SV40 promoter and the SV40 polyadenylation signal was replaced with the human growth hormone polyadenylation signal. In addition, the native polylinker was replaced with FseI, EcoRV, and AscI sites. This modified form of pAdTrach CMV was named pZyTrack. Ligation was performed using the Fast-Link™ DNA ligation and screening kit (Epicentre Technologies, Madison, Wis.) Clones containing zsig58 were identified by digestion of mini prep DNA with FseI-AscI. In order to linearize the plasmid, approximately 5 μg of the pZyTrack zsig58 plasmid was digested with PmeI. Approximately 1 μg of the linearized plasmid was cotransformed with 200 ng of supercoiled pAdEasy (He et al., supra.) into BJ5183 cells. The co-transformation was done using a Bio-Rad Gene Pulser at 2.5 kV, 200 ohms and 25 mFa. The entire co-transformation was plated on 4 LB plates containing 25 μg/ml kanamycin. The smallest colonies were picked and expanded in LB/kanamycin and recombinant adenovirus DNA identified by standard DNA miniprep procedures. Digestion of the recombinant adenovirus DNA with FseI-AscI confirmed the presence of zsig58. The recombinant adenovirus miniprep DNA was transformed into DH10B competent cells and DNA prepared using a Qiagen maxi prep kit as per kit instructions.

### Example 5

### Construct for Generating Human zsig58 Transgenic Mice

Oligonucleotides were designed to generate a PCR fragment containing a consensus Kozak sequence and the exact human zsig58 coding region. These oligonucleotides were designed with an FseI site at the 5′ end and an AscI site at the 3′ end to facilitate cloning into pMT12-8, our standard transgenic vector. PMT12-8 contains the mouse MT-1 promoter and a 5′ rat insulin II intron upstream of the FseI site.

PCR reactions were carried out with 200 ng zsig58 template (Example 1) and oligonucleotides ZC18843 (SEQ ID NO:19) and ZC18844 (SEQ ID NO:20). PCR reaction conditions were as follows: 95° C. for 5 minutes, wherein Advantage™ cDNA polymerase (Clontech) was added; 15 cycles of 95° C. for 60 seconds, 61° C. for 60 seconds, and 72° C. for 90 seconds; and 72° C. for 7 minutes. PCR products were separated by agarose gel electrophoresis and purified using a QiaQuick™ (Qiagen) gel extraction kit. The isolated, 1209 bp, DNA fragment was digested with FseI and AscI (Boerhinger-Mannheim), ethanol precipitated and ligated into pMT12-8 that was previously digested with FseI and AscI. The pMT12-8 plasmid, designed for expression of a gene of interest in transgenic mice, contains an expression cassette flanked by 10 kb of MT-1 5′ DNA and 7 kb of MT-13′ DNA. The expression cassette comprises the MT-1 promoter, the rat insulin II intron, a polylinker for the insertion of the desired clone, and the human growth hormone poly A sequence.

About one microliter of the ligation reaction was electroporated into DH10B ElectroMax™ competent cells (GIBCO BRL, Gaithersburg, Md.) according to manufacturer's direction and plated onto LB plates containing 100 μg/ml ampicillin, and incubated overnight. Colonies were picked and grown in LB media containing 100 μg/ml ampicillin. Miniprep DNA was prepared from the picked clones and screened for the zsig58 insert by restriction digestion with EcoRI, and subsequent agarose gel electrophoresis. Maxipreps of the correct pMT-zsig58 construct were performed. A SalI fragment containing with 5′ and 3′flanking sequences, the MT-1 promoter, the rat insulin II intron, zsig58 cDNA and the human growth hormone poly A sequence was prepared and used for microinjection into fertilized murine oocytes.

### Example 6

### Histopathology in Transgenic Mice Expressing zsig58

Histopathology samples described below were taken from various tissues of 4 female and 6 male transgenics (Example 5) that were either very low or low expressors. Muscle changes were evident in 2 of the low expressing transgenic females.

Tissues from thirteen mice were submitted for histology. They included ten very low to low expressing transgenic females and males, one non-expressing transgenic male, one female non-transgenic control and one male non-transgenic control. The samples included liver, heart, lung, kidney, thymus, spleen, skin, pancreas, small intestine, mesenteric lymph node, stomach, brain, salivary gland, large intestine, trachea, esophagus, thyroid, adrenal, ovary, uterus, pituitary, skeletal muscle, peripheral nerve and femur from each mouse. Using routine methods, tissue samples were fixed in 10% buffered formalin, dehydrated and cleared using a Tissue-Tek VIP automatic processor (Miles, Inc., Elkhart, IN 46515), embedded in paraffin, sectioned at 3-5 μm, and stained with hematoxylin and eosin. The sections were examined via light microscopy by an ACVP certified veterinary pathologist.

A significant finding, which was observed in two low expressing transgenic females, was a mild to moderate multifocal myopathy characterized by centralization and proliferation of myocyte nuclei with occasional fibers showing cytoplasmic basophilia indicative of regeneration. Occasional muscle fibers were swollen and had loss of striation. A low number of neutrophils were present in the interstitium adjacent to affected fibers.

### Example 7

### Expression of Human zsig58 in E. coli

A. Construction of zsig58-MBP fusion expression vector PTAP98/zsig58

An expression plasmid containing a polynucleotide encoding a human zalphall soluble receptor fused C-terminally to maltose binding protein (MBP) was constructed via homologous recombination. The polynucleotide sequence for the MBP-zalphall soluble receptor fusion polypeptide is shown in SEQ ID NO:21, with the corresponding predicted protein sequence shown in SEQ ID NO:22. A fragment of human zsig58 cDNA (SEQ ID NO:23) was isolated using PCR. Two primers were used in the production of the human zsig58 fragment in a PCR reaction: (1) Primer ZC23,288 (SEQ ID NO:24), containing 40 bp of the vector flanking sequence and 27 bp corresponding to the amino terminus of the human zsig58, and (2) primer ZC23,289 (SEQ ID NO:25), containing 40 bp of the 3′ end corresponding to the flanking vector sequence and 27 bp corresponding to the carboxyl terminus of the human zsig58. The PCR reaction conditions were as follows: 25 cycles of 94° C. for 30 seconds, 50° C. for 30 seconds, and 72° C. for 1.5 minutes; followed by 4° C. soak, run in duplicate. Two μl of the 100 μl PCR reaction were run on a 1.0% agarose gel with 1× TBE buffer for analysis, and the expected band of approximately 1140 bp fragment was seen. The remaining 90 μl of PCR reaction was combined with the second PCR tube precipitated with 400 μl of absolute ethanol to be used for recombining into the Smal cut recipient vector pTAP98 to produce the construct encoding the MBP-zsig58 fusion, as described below.

Plasmid pTAP98 was derived from the plasmids pRS316 and pMAL-c2. The plasmid pRS316 is a Saccharomyces cerevisiae shuttle vector (Hieter P. and Sikorski, R., Genetics 122:19-27, 1989). pMAL-C2 (NEB) is an E. coli expression plasmid. It carries the tac promoter driving MalE (gene encoding MBP) followed by a His tag, a thrombin cleavage site, a cloning site, and the rrnb terminator. The vector pTAP98 was constructed using yeast homologous recombination. 100 ng of EcoR1 cut pMAL-c2 was recombined with 1 μg Pvul cut pRS316, 1 μg linker, and 1 μg Scal/EcoR1 cut pRS316. The linker consisted of 4 oligos combined in a PCR reaction: ZC19,372 (SEQ ID NO:26) (100 pmol); ZC19,351 (SEQ ID NO:27) (lpmole); ZC19,352 (SEQ ID NO:28) (1 pmol); and ZC19,371 (SEQ ID NO:29) (100 pmol). Conditions were as follows: 10 cycles of 94° C. for 30 seconds, 50° C. for 30 seconds, and 72° C. for 30 seconds; followed by 4° C. soak. PCR products were concentrated via 100% ethanol precipitation.

One hundred microliters of competent yeast cells (S. cerevisiae) were combined with 10 pl of a mixture containing approximately 1 μg of the human zsig58 insert, and 100 ng of SmaI digested pTAP98 vector, and transferred to a 0.2 cm electroporation cuvette. The yeast/DNA mixture was electropulsed at 0.75 kV (5 kV/cm), infinite ohms, 25 μF. To each cuvette was added 600 μl of 1.2 M sorbitol. The yeast was then plated in two 300 μl aliquots onto two −URA D plates and incubated at 30° C.

After about 48 hours, the Ura+ yeast transformants from a single plate were resuspended in 1 ml H₂O and spun briefly to pellet the yeast cells. The cell pellet was resuspended in 1 ml of lysis buffer (2% Triton X-100, 1% SDS, 100 mM NaCl, 10 mM Tris, pH 8.0, 1 mM EDTA). Five hundred microliters of the lysis mixture was added to an Eppendorf tube containing 300 μl acid washed glass beads and 200 μl phenol-chloroform, vortexed for 1 minute intervals two or three times, followed by a 5 minute spin in a Eppendorf centrifuge at maximum speed. Three hundred microliters of the aqueous phase was transferred to a fresh tube, and the DNA precipitated with 600 μl ethanol (EtOH), followed by centrifugation for 10 minutes at 4° C. The DNA pellet was resuspended in 100 up H₂O.

Transformation of electrocompetent E. coli cells (MC1061, Casadaban et. al. J. Mol. Biol. 138, 179-207) was done with 1 μl yeast DNA prep and 40 μl of MC1061 cells. The cells were electropulsed at 2.0 kV, 25 μF and 400 ohms. Following electroporation, 0.6 ml SOC (2% BactoE Tryptone (Difco, Detroit, Mich.), 0.5% yeast extract (Difco), 10 mM NaCl, 2.5 mM KCl, 10 mM MgCl₂, 10 mM MgSO₄, 20 mM glucose) was plated in one aliquot on LB AMP plates (LB broth (Lennox), 1.8% Bacto™ Agar (Difco), 100 mg/L Ampicillin).

Individual clones harboring the correct expression construct for human zsig58 were identified by expression. Cells were grown in Superbroth II (Becton Dickinson) with 100 μg/ml of ampicillin overnight. 50 μl of the overnight culture was used to inoculate 2 ml of fresh Superbroth II +100 μg/ml ampicillin. Cultures were grown at 37° C., shaking for 2 hours. lml of the culture was induced with 1 mM IPTG. 2-4 hours later the 250 μl of each culture was mixed with 250 μl acid washed glass beads and 250 μl Thorner buffer with 5% βME and dye (8M urea, 100 mM Tris pH7.0, 10% glycerol, 2 mM EDTA, 5% SDS). Samples were vortexed for one minute and heated to 65° C. for 5-10 minutes. 20 μl were loaded per lane on a 4%-12% PAGE gel (NOVEX). Gels were run in 1× MES buffer. The positive clones were designated pTAP145 and subjected to sequence analysis to verify the sequence. The polynucleotide sequence of MBP-zsig58 fusion within pTAP145 is shown in SEQ ID NO:21.

B. Bacterial Expression of Human zsig58

One microliter of sequencing DNA was used to transform strain W3110 (ATCC). The cells were electropulsed at 2.0 kV, 25 μF and 400 ohms. Following electroporation, 0.6 ml SOC (2% Bacto™ Tryptone (Difco, Detroit, Mich.), 0.5% yeast extract (Difco), 10 mM NaCl, 2.5 mM KCl, 10 mM MgCl2, 10 mM MgSO4, 20 mM glucose) was plated in one aliquot on LB AMP plates (LB broth (Lennox), 1.8% Bacto™ Agar (Difco), 100 mg/L Ampicillin).

Individual were expressed. Cells were grown in Superbroth II (Becton Dickinson) with 100 μg/ml of ampicillin overnight. 50 μl of the overnight culture was used to inoculate 2 ml of fresh Superbroth II +100 μg/ml ampicillin. Cultures were grown at 37° C., shaking for 2 hours. 1 ml of the culture was induced with 1M IPTG. 2-4 hours later the 250 μl of each culture was mixed with 250 11 acid washed glass beads and 250 μl Thorner buffer with 5% βME and dye(8M urea, lOOmM Tris pH7.0, 10% glycerol, 2 mM EDTA, 5% SDS). Samples were vortexed for one minute and heated to 65° C. for 10 minutes. 20 μl were loaded per lane on a 4%-12% PAGE gel (NOVEX). Gels were run in 1× MES buffer. The positive clones can be grown up and used for protein purification of the MBP-zsig58 fusion protein.

### Example 8

### zsig58 Anti-Peptide Antibodies

Polyclonal anti-peptide antibodies were prepared by immunizing two female New Zealand white rabbits and 5 Balb C male mice with the peptide, huzsig58-1 (SEQ ID NO:30) a peptide from the N-terminal region of the mature zsig58 polypeptide or huzsig58-2 (SEQ ID NO:31), another zsig58 peptide. The peptides were synthesized using an Applied Biosystems Model 431A peptide synthesizer (Applied Biosystems, Inc., Foster City, Calif.) according to manufacturer's instructions. The peptides were then conjugated to the carrier protein maleimide-activated keyhole limpet hemocyanin (KLH) through cysteine residues (Pierce, Rockford, Ill.). The rabbits were each given an initial intraperitoneal (IP) injection of 200 μg of conjugated peptide in Complete Freund's Adjuvant (Pierce, Rockford, Ill.) followed by booster IP injections of 100 μg conjugated peptide in Incomplete Freund's Adjuvant every three weeks. The mice were each given an initial intraperitoneal (IP) injection of 20 μg of conjugated peptide in Complete Freund's Adjuvant followed by booster IP injections of 10 ug conjugated peptide in Incomplete Freund's Adjuvant every 2 weeks. Seven to ten days after the administration of the third booster injection, the animals were bled and the serum was collected. The rabbits were then boosted and bled every three weeks; the mice were boosted an additional 2 times followed by exsanguination.

The zsig58 peptide-specific antibodies were affinity purified from the rabbit serum using an CNBr-SEPHAROSE 4B peptide column (Pharmacia LKB) that was prepared using 10 mg of the respective peptides per gram CNBr-SEPHAROSE, followed by dialysis in PBS overnight. Peptide specific-zsig58 antibodies were characterized by an ELISA titer check using 1 μg/ml of the appropriate peptide as an antibody target.

### Example 9

### Baculovirus Expression of zSig58CEE

An expression vector, pSig5BCEE, was prepared to express zsig58 polypeptides in insect cells. pSig58CEE, was designed to express a zsig58 polypeptide with a C-terminal GLU-GLU tag (SEQ ID NO:35). This construct can be used to determine the N-terminal amino acid sequence of zSig58 after the signal peptide has been cleaved off.

A. Construction of PSig58CEE

A 1238 bp zSig58 fragment containing BglII and XbaI restriction sites on the 5′ and 3′ ends, respectively, was generated by PCR amplification from a plasmid containing zsig58 cDNA (Example 1) using primers ZC18,902 (SEQ ID NO:32) and ZC19,118 (SEQ ID NO:33). The PCR reaction conditions were as follows: 25 cycles of 94° C. for 30 seconds, 50° C. for 30 seconds, and 72° C. for 1.5 minutes; 1 cycle at 72° C. for 10 min; followed by 4° C. soak. The fragment was visualized by gel electrophoresis (1% SeaPlaque/1% NuSieve). The band was excised, diluted to 0.5% agarose with 2 mM MgCl₂, melted at 65° C. and ligated into an EcoRI/XbaI digested baculovirus expression vector, pZBV32L. The pZBV32L vector is a modification of the pFastBac1™ (Life Technologies) expression vector, where the polyhedron promoter has been removed and replaced with the late activating Basic Protein Promoter, and the coding sequence for the Glu-Glu tag as well as a stop signal is inserted at the 3′ end of the multiple cloning region). About 25 nanograms of the restriction digested zsig58 insert and about 32 ng of the corresponding vector were ligated overnight at 16° C. The ligation mix was diluted 3 fold in TE (10 mM Tris-HCl, pH 7.5 and 1 mM EDTA) and 4 fmol of the diluted ligation mix was transformed into DH5α Library Efficiency competent cells (Life Technologies) according to manufacturer's direction by heat shock for 45 seconds in a 42° C. waterbath. The transformed DNA and cells were diluted in 450 μl of SOC media (2% Bacto Tryptone, 0.5% Bacto Yeast Extract, 10 ml 1M NaCl, 1.5 mM KCl, 10 mM MgCl₂, 10 MM MgSO₄ and 20 mM glucose) and plated onto LB plates containing 100 μg/ml ampicillin. Clones were analyzed by restriction digests and 1 μl of the positive clone was transformed into 20 μl DH10Bac Max Efficiency competent cells (GIBCO-BRL, Gaithersburg, Md.) according to manufacturer's instruction, by heat shock for 45 seconds in a 42° C. waterbath. The ligated DNA was diluted in 980 μl SOC media (2% Bacto Tryptone, 0.5% Bacto Yeast Extract, 10 ml 1M NaCl, 1.5 mM KCl, 10 MM MgCl₂, 10 mM MgSO₄ and 20 mM glucose) and plated onto Luria Agar plates containing 50 μg/ml kanamycin, 7 μg/ml gentamicin, 10 μg/ml tetracycline, IPTG and Bluo Gal. The cells were incubated for 48 hours at 37° C. A color selection was used to identify those cells having virus that had incorporated into the plasmid (referred to as a “bacmid”). Those colonies, which were white in color, were picked for analysis. Bacmid DNA was isolated from positive colonies using the QiaVac Miniprep8 system (Qiagen) according the manufacturer's directions. Clones were screened for the correct insert by amplifying DNA using primers to the transposable element in the bacmid via PCR using primers ZC447 (SEQ ID NO:34) and ZC976 (SEQ ID NO:7). The PCR reaction conditions were as follows: 35 cycles of 94° C. for 45 seconds, 50° C. for 45 seconds, and 72° C. for 5 minutes; 1 cycle at 72° C. for 10 min.; followed by 4° C. soak. The PCR product was run on a 1% agarose gel to check the insert size. Those having the correct insert were used to transfect Spodoptera frugiperda (Sf9) cells.

B. Transfection

Sf9 cells were seeded at 5×10⁶ cells per 35 mm plate and allowed to attach for 1 hour at 27° C. Five microliters of bacmid DNA was diluted with 100 μl Sf-900 II SFM (Life Technologies). Six μl of CellFECTIN Reagent (Life Technologies) was diluted with 100 μl Sf-900 II SFM. The bacmid DNA and lipid solutions were gently mixed and incubated 30-45 minutes at room temperature. The media from one plate of cells were aspirated, the cells were washed 1× with 2 ml fresh Sf-900 II SFM media. Eight hundred microliters of Sf-900 II SFM was added to the lipid-DNA mixture. The wash media was aspirated and the DNA-lipid mix added to the cells. The cells were incubated at 27° C. for 4-5 hours. The DNA-lipid mix was aspirated and 2 ml of Sf-900 II media was added to each plate. The plates were incubated at 27° C., 90% humidity, for 96 hours after which the virus was harvested.

C. Primary Amplification

Sf9 cells were grown in 50 ml Sf-900 II SFM in a 125 ml shake flask to an approximate density of 0.41-0.52×10⁵ cells/ml. They were then infected with 100 μl of the virus stock from above and incubated at 27° C. for 2-3 days after which time the virus was harvested according to standard methods known in the art.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polynucleotide encoding a zsig58 polypeptide comprising a sequence of amino acid residues that is at least 90% identical to an amino acid sequence selected from the group consisting of:
(a) the amino acid sequence as shown in SEQ ID NO:2 from amino acid number 141 (Cys) to amino acid number 402 (Lys);
(b) the amino acid sequence as shown in SEQ ID NO:2 from amino acid number 26 (Thr) to amino acid number 402 (Lys); and
(c) the amino acid sequence as shown in SEQ ID NO:2 from amino acid number 1 (Met) to amino acid number 402 (Lys),
wherein the amino acid percent identity is determined using a FASTA program with ktup=1, gap opening penalty=10, gap extension penalty=1, and substitution matrix=BLOSUM62, with other parameters set as default.

2. An isolated polynucleotide according to claim 1, wherein the polynucleotide is selected from the group consisting of:
(a) a polynucleotide sequence as shown in SEQ ID NO:1 from nucleotide 505 to nucleotide 1290;
(b) a polynucleotide sequence as shown in SEQ ID NO:1 from nucleotide 160 to nucleotide 1290; and
(c) a polynucleotide sequence as shown in SEQ ID NO:l from nucleotide 85 to nucleotide 1290.

3. An isolated polynucleotide sequence according to claim 1, wherein the polynucleotide comprises nucleotide 1 to nucleotide 1206 of SEQ ID NO:5.

4. An isolated polynucleotide according to claim 1, wherein the zsig58 polypeptide comprises a sequence of amino acid residues that is identical to an amino acid sequence selected from the group consisting of:
(a) the amino acid sequence as shown in SEQ ID NO:2 from amino acid number 141 (Cys) to amino acid number 402 (Lys);
(b) the amino acid sequence as shown in SEQ ID NO:2 from amino acid number 26 (Thr) to amino acid number 402 (Lys); and
(c) the amino acid sequence as shown in SEQ ID NO:2 from amino acid number 1 (Met) to amino acid number 402 (Lys).

5. An isolated polynucleotide according to claim 4, wherein the zsig58 polypeptide consists of a sequence of amino acid residues as shown in SEQ ID NO:2 from amino acid number 26 (Thr) to amino acid number 402 (Lys).

6. An expression vector comprising the following operably linked elements:
a transcription promoter;
a DNA segment encoding a zsig58 polypeptide as shown in SEQ ID NO:2 from amino acid number 26 (Thr) to amino acid number 402 (Lys); and
a transcription terminator.

7. An expression vector according to claim 6, further comprising a secretory signal sequence operably linked to the DNA segment.

8. A cultured cell into which has been introduced an expression vector according to claim 6, wherein the cell expresses a polypeptide encoded by the DNA segment.

9. A DNA construct encoding a fusion protein, the DNA construct comprising:
a first DNA segment encoding a polypeptide selected from the group consisting of:
(a) the amino acid sequence of SEQ ID NO: 2 from residue number 1 (Met), to residue 25 (Cys);
(b) the amino acid sequence of SEQ ID NO: 2 from residue number 26 (Thr), to residue number 140 (Ser);
(c) the amino acid sequence of SEQ ID NO: 2 from residue number 141 (Cys) to amino acid residue 402 (Lys); and
(d) the amino acid sequence of SEQ ID NO: 2 from residue number 26 (Thr), to residue number 402 (Lys); and
at least one other DNA segment encoding an additional polypeptide,
wherein the first and other DNA segments are connected in-frame; and
encode the fusion protein.

10. A fusion protein produced by a method comprising:
culturing a host cell into which has been introduced a vector comprising the following operably linked elements:
(a) a transcriptional promoter;
(b) a DNA construct encoding a fusion protein according to claim 9; and
(c) a transcriptional terminator; and
recovering the protein encoded by the DNA segment.

11. An isolated polypeptide comprising a sequence of amino acid residues that is at least 90% identical to an amino acid sequence selected from the group consisting of:
(a) the amino acid sequence as shown in SEQ ID NO:2 from amino acid number 141 (Cys) to amino acid number 402 (Lys);
(b) the amino acid sequence as shown in SEQ ID NO:2 from amino acid number 26 (Thr) to amino acid number 402 (Lys); and
(c) the amino acid sequence as shown in SEQ ID NO:2 from amino acid number 1 (Met) to amino acid number 402 (Lys),
wherein the amino acid percent identity is determined using a FASTA program with ktup=1, gap opening penalty=10, gap extension penalty=1, and substitution matrix=BLOSUM62, with other parameters set as default.

12. An isolated polypeptide according to claim 11, wherein the polypeptide further contains motifs 1 through 7 spaced apart from N-terminus to C-terminus in a configuration selected from the group consisting of:
(a) M1-{46}-M2-{37-41}-M3; and
(b) M4-{48-52}-M5-{49}-Ml-{1}-M6-{39}-M2-{37-41}-M3-{4}-M7.

13. An isolated polypeptide according to claim 11, wherein the polypeptide comprises a sequence of amino acid residues that is identical to an amino acid sequence selected from the group consisting of:
(a) the amino acid sequence as shown in SEQ ID NO:2 from amino acid number 141 (Cys) to amino acid number 402 (Lys);
(b) the amino acid sequence as shown in SEQ ID NO:2 from amino acid number 26 (Thr) to amino acid number 402 (Lys); and
(c) the amino acid sequence as shown in SEQ ID NO:2 from amino acid number 1 (Met) to amino acid number 402 (Lys).

14. An isolated polypeptide according to claim 13, wherein. the sequence of amino acid residues is as shown in SEQ ID NO:2 from amino acid number 26 (Thr) to amino acid number 402 (Lys).

15. A method of producing a zsig58 polypeptide comprising:
culturing a cell according to claim 8; and
isolating the zsig58 polypeptide produced by the cell.

16. A method of detecting, in a test sample, the presence of a modulator of zsig58 protein activity, comprising:
transfecting a zsig58-responsive cell, with a reporter gene construct that is responsive to a zsig58-stimulated cellular pathway; and
producing a zsig58 polypeptide by the method of claim 14; and
adding the zsig58 polypeptide to the cell, in the presence and absence of a test sample; and
comparing levels of response to the zsig58 polypeptide, in the presence and absence of the test sample, by a biological or biochemical assay; and
determining from the comparison, the presence of the modulator of zsig58 activity in the test sample.

17. A method of producing an antibody to zsig58 polypeptide comprising the following steps in order:
inoculating an animal with a polypeptide selected from the group consisting of:
(a) a polypeptide consisting of 9 to 402 amino acids, wherein the polypeptide is identical to a contiguous sequence of amino acids in SEQ ID NO:2 from amino acid number 26 (Thr) to amino acid number 402 (Lys);
(b) the amino acid sequence of SEQ ID NO: 2 from residue number 26 (Thr), to residue number 140 (Ser);
(c) a polypeptide according to claim 11; and
wherein the polypeptide elicits an immune response in the animal; and
isolating the antibody from the animal.

18. An antibody produced by the method of claim 17, which binds to a zsig58 polypeptide.

19. The antibody of claim 18, wherein the antibody is a monoclonal antibody.

20. An antibody which specifically binds to a polypeptide of claim 11.


SEQUENCE LISTING (35 sequences)

SEQ ID NO: 1 - DNA, 2479 bp, Homo sapiens
  CDS (85)...(1290)
agcaggagagcaccaccggagcccttgagacatccttgagaagagccacagcataagagactgccctgcttggtgttttgcaggatgatggtggcccttcgaggagcttctgcattgctggttctgttccttgcagcttttctgcccccgccgcagtgtacccaggacccagccatggtgcattacatctaccagcgctttcgagtcttggagcaagggctggaaaaatgtacccaagcaacgagggcatacattcaagaattccaagagttctcaaaaaatatatctgtcatgctgggaagatgtcagacctacacaagtgagtacaagagtgcagtgggtaacttggcactgagagttgaacgtgcccaacgggagattgactacatacaataccttcgagaggctgacgagtgcatcgaatcagaggacaagacactggcagaaatgttgctccaagaagctgaagaagagaaaaagatccggactctgctgaatgcaagctgtgacaacatgctgatgggcataaagtctttgaaaatagtgaagaagatgatggacacacatggctcttggatgaaagatgctgtctataactctccaaaggtgtacttattaattggatccagaaacaacactgtttgggaatttgcaaacatacgggcattcatggaggataacaccaagccagctccccggaagcaaatcctaacactttcctggcagggaacaggccaagtgatctacaaaggttttctattttttcataaccaagcaacttctaatgagataatcaaatataacctgcagaagaggactgtggaagatcgaatgctgctcccaggaggggtaggccgagcattggtttaccagcactccccctcaacttacattgacctggctgtggatgagcatgggctctgggccatccactctgggccaggcacccatagccatttggttctcacaaagattgagccgggcacactgggagtggagcattcatgggataccccatgcagaagccaggatgctgaagcctcattcctcttgtgtggggttctctatgtggtctacagtactgggggccagggccctcatcgcatcacctgcatctatgatccactgggcactatcagtgaggaggacttgcccaacttgttcttccccaagagaccaagaagtcactccatgatccattacaaccccagagataagcagctctatgcctggaatgaaggaaaccagatcatttacaaactccagacaaagagaaagctgcctctgaagtaatgcattacagctgtgagaaagagcactgtggctttggcagctgttctacaggacagtgaggctatagccccttcacaatatagtatccctctaatcacacacaggaagagtgtgtagaagtggaaatacgtatgcctcctttcccaaatgtcactgccttaggtatcttccaagagcttagatgagagcatatcatcaggaaagtttcaacaatgtccattactcccccaaacctcctggctctcaaggatgaccacattctgatacagcctacttcaagccttttgttttactgctccccagcatttactgtaactctgccatcttccctcccacaattagagttgtatgccagcccctaatattcaccactggcttttctctcccctggcctttgctgaagctcttccctctttttcaaatgtctattgatattctcccattttcactgcccaactaaaatactattaatatttctttcttttcttttcttttttttgagacaaggtctcactatgttgcccaggctggtctcaaactccagagctcaagagatcctcctgcctcagcctcctaagtacctgggattacaggcatgtgccaccacacctggcttaaaatactatttcttattgaggtttaacctctatttcccctagccctgtccttccactaagcttggtagatgtaataataaagtgaaaatattaacatttgaatatcgctttccaggtgtggagtgtttgcacatcattgaattctcgtttcacctttgtgaaacatgcacaagtctttacagctgtcattctagagtttaggtgagtaacacaattacaaagtgaaagatacagctagaaaatactacaaatcccatagtttttccattgcccaaggaagcatcaaatacgtatgtttgttcacctactcttatagtcaatgcgttcatcgtttcagcctaaaaataatagtctgtccctttagccagttttcatgtctgcacaagacctttcaataggcctttcaaatgataattcctccagaaaaccagtctaagggtgaggaccccaactctagcctcctcttgtcttgctgtcctctgtttctctctttctgctttaaattcaataaaagtgacactgagcaaataacctcatcaggttatatttgcccacataccctaagcaca

SEQ ID NO: 2 - protein, 402 aa, Homo sapiens
MMVALRGASALLVLFLAAFLPPPQCTQDPAMVHYIYQRFRVLEQGLEKCTQATRAYIQEFQEFSKNISVMLGRCQTYTSEYKSAVGNLALRVERAQREIDYIQYLREADECIESEDKTLAEMLLQEAEEEKKIRTLLNASCDNMLMGIKSLKIVKKMMDTHGSWMKDAVYNSPKVYLLIGSRNNTVWEFANIRAFMEDNTKPAPRKQILTLSWQGTGQVIYKGFLFFHNQATSNEIIKYNLQKRTVEDRMLLPGGVGRALVYQHSPSTYIDLAVDEHGLWAIHSGPGTHSHLVLTKIEPGTLGVEHSWDTPCRSQDAEASFLLCGVLYVVYSTGGQGPHRITCIYDPLGTISEEDLPNLFFPKRPRSHSMIHYNPRDKQLYAWNEGNQIIYKLQTKRKLPLK

SEQ ID NO: 3 - protein, 457 aa, Rattus norvegicus
MQPARKLLSLLVLLVMGTELTQVLPTNPEESWQVYSSAQDSEGRCICTVVAPQQTMCSRDARTKQLRQLLEKVQNMSQSIEVLDRRTQRDLQYVEKMENQMKGLESKFRQVEESHKQHLARQFKAIKAKMDELRPLIPVLEEYKADAKLVLQFKEEVQNLTSVLNELQEEIGAYDYDELQSRVSNLEERLRACMQKLACGKLTGISDPVTVKTSGSRFGSWMTDPLAPEGDNRVWYMDGYHNNRFVREYKSMVDFMNTDNFTSHRLPHPWSGTGQVVYNGSIYFNKFQSHIIIRFDLKTETILKTRSLDYAGYNNMYHYAWGGHSDIDLMVDENGLWAVYATNQNAGNIVISKLDPVSLQILQTWNTSYPKRSAGEAFIICGTLYVTNGYSGGTKVHYAYQTNASTYEYIDIPFQNKYSHISMLDYNPKDRALYAWNNGHQTLYNVTLFHVIRSDEL

SEQ ID NO: 4 - protein, 504 aa, Homo sapiens
MRFFCARCCSFGPEMPAVQLLLLACLVWDVGARTAQLRKANDQSGRCQYTFSVASPNESSCPEQSQAMSVIHNLQRDSSTQRLDLEATKARLSSLESLLHQLTLDQAARPQETQEGLQRELGTLRRERDQLETQTRELETAYSNLLRDKSVLEEEKKRLRQENENLARRLESSSQEVARLRRGQCPQTRDTARAVPPGSREVSTWNLDTLAFQELKSELTEVPASRILKESPSGYLRSGEGDTGCGELVWVGEPLTLRTAETITGKYGVWMRDPKPTYPYTQETTWRIDTVGTDVRQVFEYDLISQFMQGYPSKVHILPRPLESTGAVVYSGSLYFQGAESRTVIRYELNTETVKAEKEIPGAGYHGQFPYSWGGYTDIDLAVDEAGLWVIYSTDEAKGAIVLSKLNPENLELEQTWETNIRKQSVANAFIICGTLYTVSSYTSADATVNFAYDTGTGISKTLTIPFKNRYKYSSMIDYNPLEKKLFAWDNLNMVTYDIKLSKM

SEQ ID NO: 5 - DNA, 1206 bp, Artificial Sequence
  Degenerate sequence derived from zsig58 amino acid sequence
atgatggtngcnytnmgnggngcnwsngcnytnytngtnytnttyytngcngcnttyytnccnccnccncartgyacncargayccngcnatggtncaytayathtaycarmgnttymgngtnytngarcarggnytngaraartgyacncargcnacnmgngcntayathcargarttycargarttywsnaaraayathwsngtnatgytnggnmgntgycaracntayacnwsngartayaarwsngcngtnggnaayytngcnytnmgngtngarmgngcncarmgngarathgaytayathcartayytnmgngargcngaygartgyathgarwsngargayaaracnytngcngaratgytnytncargargcngargargaraaraarathmgnacnytnytnaaygcnwsntgygayaayatgytnatgggnathaarwsnytnaarathgtnaaraaratgatggayacncayggnwsntggatgaargaygcngtntayaaywsnccnaargtntayytnytnathggnwsnmgnaayaayacngtntgggarttygcnaayathmgngcnttyatggargayaayacnaarccngcnccnmgnaarcarathytnacnytnwsntggcarggnacnggncargtnathtayaarggnttyytnttyttycayaaycargcnacnwsnaaygarathathaartayaayytncaraarmgnacngtngargaymgnatgytnytnccnggnggngtnggnmgngcnytngtntaycarcaywsnccnwsnacntayathgayytngcngtngaygarcayggnytntgggcnathcaywsnggnccnggnacncaywsncayytngtnytnacnaarathgarccnggnacnytnggngtngarcaywsntgggayacnccntgymgnwsncargaygcngargcnwsnttyytnytntgyggngtnytntaygtngtntaywsnacnggnggncarggnccncaymgnathacntgyathtaygayccnytnggnacnathwsngargargayytnccnaayytnttyttyccnaarmgnccnmgnwsncaywsnatgathcaytayaayccnmgngayaarcarytntaygcntggaaygarggnaaycarathathtayaarytncaracnaarmgnaarytnccnytnaar

SEQ ID NO: 6 - protein, 8 aa, Artificial Sequence
  Peptide affinity tag
DYKDDDDK

SEQ ID NO: 7 - DNA, 18 bp, Artificial Sequence
  Oligonucleotide primer ZC976
cgttgtaaaacgacggcc

SEQ ID NO: 8 - DNA, 25 bp, Artificial Sequence
  Oligonucleotide primer ZC6768
gcaattaaccctcactaaagggaac

SEQ ID NO: 9 - DNA, 20 bp, Artificial Sequence
  Oligonucleotide primer ZC17601
tgatgcttccttgggcaatg

SEQ ID NO: 10 - DNA, 20 bp, Artificial Sequence
  Oligonucleotide primer ZC17602
agaggacaagacactggcag

SEQ ID NO: 11 - DNA, 21 bp, Artificial Sequence
  Oligonucleotide primer ZC17766
acaggacagtgaggctatagc

SEQ ID NO: 12 - DNA, 21 bp, Artificial Sequence
  Oligonucleotide primer ZC17767
agcctcactgtcctgtagaac

SEQ ID NO: 13 - DNA, 20 bp, Artificial Sequence
  Oligonucleotide primer ZC17676
ttacattgacctggctgtgg

SEQ ID NO: 14 - DNA, 19 bp, Artificial Sequence
  Oligonucleotide primer ZC17677
tgggcaacatagtgagacc

SEQ ID NO: 15 - DNA, 21 bp, Artificial Sequence
  Oligonucleotide primer ZC17852
acaaggtctcactatgttgcc

SEQ ID NO: 16 - DNA, 19 bp, Artificial Sequence
  Oligonucleotide primer ZC17854
atccacagccaggtcaatg

SEQ ID NO: 17 - DNA, 18 bp, Artificial Sequence
  Oligonucleotide primer ZC18333
tccggactctgctgaatg

SEQ ID NO: 18 - DNA, 18 bp, Artificial Sequence
  Oligonucleotide primer ZC18334
agctggcttggtgttatc

SEQ ID NO: 19 - DNA, 32 bp, Artificial Sequence
  Oligonucleotide primer ZC18843
gtatacggccggccaccatgatggtggccctt

SEQ ID NO: 20 - DNA, 32 bp, Artificial Sequence
  Oligonucleotide primer ZC18844
cgtacgggcgcgccttacttcagaggcagctt

SEQ ID NO: 21 - DNA, 2298 bp, Artificial Sequence
  Polynucleotide encoding a MBP-zsig58 fusion polypeptide
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcccaccatcaccatcaccacgcgaattcggtaccgctggttccgcgtggatccacccaggacccagccatggtgcattacatctaccagcgctttcgagtcttggagcaagggctggaaaaatgtacccaagcaacgagggcatacattcaagaattccaagagttctcaaaaaatatatctgtcatgctgggaagatgtcagacctacacaagtgagtacaagagtgcagtgggtaacttggcactgagagttgaacgtgcccaacgggagattgactacatacaataccttcgagaggctgacgagtgcatcgaatcagaggacaagacactggcagaaatgttgctccaagaagctgaagaagagaaaaagatccggactctgctgaatgcaagctgtgacaacatgctgatgggcataaagtctttgaaaatagtgaagaagatgatggacacacatggctcttggatgaaagatgctgtctataactctccaaaggtgtacttattaattggatccagaaacaacactgtttgggaatttgcaaacatacgggcattcatggaggataacaccaagccagctccccggaagcaaatcctaacactttcctggcagggaacaggccaagtgatctacaaaggttttctattttttcataaccaagcaacttctaatgagataatcaaatataacctgcagaagaggactgtggaagatcgaatgctgctcccaggaggggtaggccgagcattggtttaccagcactccccctcaacttacattgacctggctgtggatgagcatgggctctgggccatccactctgggccaggcacccatagccatttggttctcacaaagattgagccgggcacactgggagtggagcattcatgggataccccatgcagaagccaggatgctgaagcctcattcctcttgtgtggggttctctatgtggtctacagtactgggggccagggccctcatcgcatcacctgcatctatgatccactgggcactatcagtgaggaggacttgcccaacttgttcttccccaagagaccaagaagtcactccatgatccattacaaccccagagataagcagctctatgcctggaatgaaggaaaccagatcatttacaaactccagacaaagagaaagctgcctctgaagtaa

SEQ ID NO: 22 - protein, 765 aa, Artificial Sequence [sequence omitted]

SEQ ID NO: 23 - DNA, 1134 bp, Homo sapiens
acccaggacccagccatggtgcattacatctaccagcgctttcgagtcttggagcaagggctggaaaaatgtacccaagcaacgagggcatacattcaagaattccaagagttctcaaaaaatatatctgtcatgctgggaagatgtcagacctacacaagtgagtacaagagtgcagtgggtaacttggcactgagagttgaacgtgcccaacgggagattgactacatacaataccttcgagaggctgacgagtgcatcgaatcagaggacaagacactggcagaaatgttgctccaagaagctgaagaagagaaaaagatccggactctgctgaatgcaagctgtgacaacatgctgatgggcataaagtctttgaaaatagtgaagaagatgatggacacacatggctcttggatgaaagatgctgtctataactctccaaaggtgtacttattaattggatccagaaacaacactgtttgggaatttgcaaacatacgggcattcatggaggataacaccaagccagctccccggaagcaaatcctaacactttcctggcagggaacaggccaagtgatctacaaaggttttctattttttcataaccaagcaacttctaatgagataatcaaatataacctgcagaagaggactgtggaagatcgaatgctgctcccaggaggggtaggccgagcattggtttaccagcactccccctcaacttacattgacctggctgtggatgagcatgggctctgggccatccactctgggccaggcacccatagccatttggttctcacaaagattgagccgggcacactgggagtggagcattcatgggataccccatgcagaagccaggatgctgaagcctcattcctcttgtgtggggttctctatgtggtctacagtactgggggccagggccctcatcgcatcacctgcatctatgatccactgggcactatcagtgaggaggacttgcccaacttgttcttccccaagagaccaagaagtcactccatgatccattacaaccccagagataagcagctctatgcctggaatgaaggaaaccagatcatttacaaactccagacaaagagaaagctgcctctgaagtaa

SEQ ID NO: 24 - DNA, 67 bp, Artificial Sequence
  Oligonucleotide primer ZC23288
tcaccacgcgaattcggtaccgctggttccgcgtggatccacccaggacccagccatggtgcattac

SEQ ID NO: 25 - DNA, 67 bp, Artificial Sequence
  Oligonucleotide primer ZC23289
tctgtatcaggctgaaaatcttatctcatccgccaaaacattacttcagaggcagctttctctttgt

SEQ ID NO: 26 - DNA, 40 bp, Artificial Sequence
  Oligonucleotide primer ZC19372
tgtcgatgaagccctgaaagacgcgcagactaattcgagc

SEQ ID NO: 27 - DNA, 60 bp, Artificial Sequence
  Oligonucleotide primer ZC19351
acgcgcagactaattcgagctcccaccatcaccatcaccacgcgaattcggtaccgctgg

SEQ ID NO: 28 - DNA, 60 bp, Artificial Sequence
  Oligonucleotide primer ZC19352
actcactatagggcgaattgcccgggggatccacgcggaaccagcggtaccgaattcgcg

SEQ ID NO: 29 - DNA, 42 bp, Artificial Sequence
  Oligonucleotide primer ZC19731
acggccagtgaattgtaatacgactcactatagggcgaattg

SEQ ID NO: 30 - protein, 22 aa, Artificial Sequence
  Synthesized human zsig58 peptide, huzsig58-1
DPAMVHYIYQRFRVLEQGLEKC

SEQ ID NO: 31 - protein, 30 aa, Artificial Sequence
  Synthesized human zsig58 peptide, huzsig58-2
IQYLREADECIESEDKTLAEMLLQEAEEEK

SEQ ID NO: 32 - DNA, 29 bp, Artificial Sequence
  Oligonucleotide primer ZC18902
gtgtttagatctatgatggtggcccttcg

SEQ ID NO: 33 - DNA, 30 bp, Artificial Sequence
  Oligonucleotide primer ZC19118
cacagctgtaatgtctagattcagaggcag

SEQ ID NO: 34 - DNA, 17 bp, Artificial Sequence
  Oligonucleotide primer ZC447
taacaatttcacacagg

SEQ ID NO: 35 - protein, 6 aa, Artificial Sequence
  Glu-Glu tag peptide
EYMPME